SEQUENCE LISTING (46 sequences)

SEQ ID NO: 1 - DNA, 12 bp, Artificial Sequence
  Primer
agaatgtccgta

SEQ ID NO: 2 - DNA, 12 bp, Artificial Sequence
  Primer
ccgcagttagat

SEQ ID NO: 3 - DNA, 12 bp, Artificial Sequence
  Primer
cagtgggagttt

SEQ ID NO: 4 - DNA, 12 bp, Artificial Sequence
  Primer
tctatggaccct

SEQ ID NO: 5 - DNA, 12 bp, Artificial Sequence
  Primer
ttcattctgggg

SEQ ID NO: 6 - DNA, 12 bp, Artificial Sequence
  Primer
ccgtcttttctg

SEQ ID NO: 7 - DNA, 12 bp, Artificial Sequence
  Primer
tggcctattggc

SEQ ID NO: 8 - DNA, 12 bp, Artificial Sequence
  Primer
gtcatgcctgga

SEQ ID NO: 9 - DNA, 12 bp, Artificial Sequence
  Primer
gacctgcgatct

SEQ ID NO: 10 - DNA, 12 bp, Artificial Sequence
  Primer
tggcctcttgga

SEQ ID NO: 11 - DNA, 12 bp, Artificial Sequence
  Primer
ggtttcccagga

SEQ ID NO: 12 - DNA, 12 bp, Artificial Sequence
  Primer
tcgtccggagat

SEQ ID NO: 13 - DNA, 12 bp, Artificial Sequence
  Primer
cgcttcgtagca

SEQ ID NO: 14 - DNA, 12 bp, Artificial Sequence
  Primer
ggcttcgaatcg

SEQ ID NO: 15 - DNA, 12 bp, Artificial Sequence
  Primer
gatgagctaaaa

SEQ ID NO: 16 - DNA, 12 bp, Artificial Sequence
  Primer
gagcaggaatat

SEQ ID NO: 17 - DNA, 12 bp, Artificial Sequence
  Primer
cttaggttacgt

SEQ ID NO: 18 - DNA, 12 bp, Artificial Sequence
  Primer
gtggatctgaat

SEQ ID NO: 19 - DNA, 12 bp, Artificial Sequence
  Primer
cctatcccaaca

SEQ ID NO: 20 - DNA, 12 bp, Artificial Sequence
  Primer
gagactaccgaa

SEQ ID NO: 21 - DNA, 12 bp, Artificial Sequence
  Primer
gacagcgtccta

SEQ ID NO: 22 - DNA, 12 bp, Artificial Sequence
  Primer
cttgagcgtatt

SEQ ID NO: 23 - DNA, 12 bp, Artificial Sequence
  Primer
actgagatagca

SEQ ID NO: 24 - DNA, 12 bp, Artificial Sequence
  Primer
gagagacgatta

SEQ ID NO: 25 - DNA, 12 bp, Artificial Sequence
  Primer
gacgcccattat

SEQ ID NO: 26 - DNA, 12 bp, Artificial Sequence
  Primer
gacggttctaca

SEQ ID NO: 27 - DNA, 12 bp, Artificial Sequence
  Primer
gatggtccgttt

SEQ ID NO: 28 - DNA, 12 bp, Artificial Sequence
  Primer
ttcaccaacgag

SEQ ID NO: 29 - DNA, 12 bp, Artificial Sequence
  Primer
caggtgtgggtt

SEQ ID NO: 30 - DNA, 12 bp, Artificial Sequence
  Primer
attggtgcagaa

SEQ ID NO: 31 - DNA, 12 bp, Artificial Sequence
  Primer
aaggcgtgttta

SEQ ID NO: 32 - DNA, 12 bp, Artificial Sequence
  Primer
tattgggattgg

SEQ ID NO: 33 - DNA, 12 bp, Artificial Sequence
  Primer
aacatctccggg

SEQ ID NO: 34 - DNA, 12 bp, Artificial Sequence
  Primer
atcattggcgaa

SEQ ID NO: 35 - DNA, 12 bp, Artificial Sequence
  Primer
ttgagtagttgc

SEQ ID NO: 36 - DNA, 12 bp, Artificial Sequence
  Primer
tacgccggaata

SEQ ID NO: 37 - DNA, 12 bp, Artificial Sequence
  Primer
cctgaggtagct

SEQ ID NO: 38 - DNA, 12 bp, Artificial Sequence
  Primer
gccgcttcagct

SEQ ID NO: 39 - DNA, 12 bp, Artificial Sequence
  Primer
atctaaaccacg

SEQ ID NO: 40 - DNA, 12 bp, Artificial Sequence
  Primer
agagctgaagta

SEQ ID NO: 41 - DNA, 12 bp, Artificial Sequence
  Primer
gccttcgttacg

SEQ ID NO: 42 - DNA, 12 bp, Artificial Sequence
  Primer
agggctctaggc

SEQ ID NO: 43 - DNA, 12 bp, Artificial Sequence
  Primer
ttgcataatcgt

SEQ ID NO: 44 - DNA, 12 bp, Artificial Sequence
  Primer
ggcagatatcat

SEQ ID NO: 45 - DNA, 12 bp, Artificial Sequence
  Primer
tccaagctacca

SEQ ID NO: 46 - DNA, 12 bp, Artificial Sequence
  Primer
taacaaccgagc

(19) United States
(12) Patent Application Publication
(10) Pub. No.: US 2003/0180716 A1
(43) Pub. Date: Sep. 25, 2003

(54) Microbe indentifying method, microbe identifying apparatus, method for creating database for microbe identification, microbe identifying program, and recording medium on which the same is recorded

(76) Inventor: Takakazu Inoue, Ushiku-shi (JP)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(21) Appl. No.: 10/240,580

(22) PCT Filed: Mar. 27, 2001
(86) PCT No.: PCT/JP01/02516

(30) Foreign Application Priority Data
Mar. 31, 2000 (JP) 2000-099482

(51) Int. Cl. C12Q 1/70; C12Q 1/68; G06F 19/00; G01N 33/48; G01N 33/50

(52) U.S. Cl. 435/5; 435/6; 702/20

(57) ABSTRACT

A microorganism discriminating method includes a step S1 of conducting an analytical experiment on microorganisms by a SSC-PCR method, a step S2 of capturing an electrophoretic image obtained by the analytical experiment, a step S3 of detecting a band from image data, a step S4 of carrying out image correction, a step S5 of measuring the position and the intensity of the band, a step S6 of creating a list of band data by collecting the results of measurement, a step S7 of searching database by employing the band data list, and a step S8 of displaying the results.

### TECHNICAL FIELD

The present invention relates to a microorganism discriminating method, a microorganism discriminating apparatus, a method of creating database for microorganism discrimination, a microorganism discriminating program and a recording medium on which such a program is recorded.

### BACKGROUND ART

In recent, years, organic waste processors for composting organic wastes (so-called kitchen refuse or garbage) discharged from houses and the like are now under active researches and development. In such organic waste processors, microorganism groups such as bacteria and protozoa decompose organic matters to form compost.

In a composting process (a process of decomposing organic matters) by the organic waste processor, the degree of composting is evaluated by monitoring temperatures and the like. The state of the organic waste processor is adjusted to create the compost of high quality on the basis of the evaluation.

In order to create the compost of higher quality, however, it is necessary to obtain information as to a microorganism group (at least the types of microorganisms) functioning within the organic waste processor. Such information on the microorganism group is also required to achieve excellent control on the decomposition of garbage by microorganisms. Also, it is important to acquire information as to the microorganism group in the soil in order to improve the soil by adding the created compost.

As a method of obtaining the information on the microorganism group, for example, the information on a bacteria group, such a method or the like has conventionally been applied that individual bacteria contained in the bacteria group are isolated to undergo a biochemical examination. This method is, however, time-consuming and inappropriate for the examination of bacteria that are hard to be isolated.

Alternatively, such a method is considered that obtains the information on a microorganism group by conducting a DNA analysis. Amplification of DNA is required for conducting the DNA analysis. As a method of amplifying the DNA, a PCR (polymerase chain reaction) is employed (U.S. Pat. Nos. 4,683,195, 4,683,202, 4,965,188, 5,038,852 and 5,333,675). According to the PCR method, by employing a primer that has a base sequence complementary with base sequences on opposite ends of DNA to be amplified (template DNA) and a heat-resistant DNA polymerase, and repeating a cycle of three stages, a thermal denaturation step, an annealing (heat treatment) step and an extension reaction step, it is made possible to amplify DNA fragments that are substantially the same as the template DNA. The use of the PCR method enables amplifying a predetermined fragment in a single DNA of a slight amount of bacteria up to, e.g., one hundred thousand to one million times.

However, at least the base sequences on the opposite ends of one region of the template need be known in order for the use of the PCR method. Therefore, according to the conventional PCR method, if the types and base sequences of microorganisms functioning in the organic waste processors and microorganisms existing in the soil are unknown, then it is impossible to amplify the DNA fragments of those microorganisms.

Thus, a RAPD (random amplified polymorphic DNA) method or an AP-PCR (arbitrarily primed-polymerase chain reaction) method is proposed that amplifies a single type of DNA to many types of DNA fragments at a time by employing a single primer without any information on base sequences. In such a method, sequence specificity at bonding of the primer is degraded by decreasing an annealing temperature of the primer at the PCR and further increasing the concentration of magnesium ions in a reaction solution. Then, the primer is bonded to chromosome DNA of a microorganism along with mismatching, and the DNA fragments are replicated.

According to the RAPD method or AP-PCR method, a large amount of some DNA fragments are amplified by a single primer without any information as to the base sequence of the DNA to be amplified. A DNA fingerprint is obtained by separating the amplified DNA fragments by gel electrophoresis. Analyzing this DNA fingerprint enables analysis of the microorganism.

On the other hand, when the conventional RAPD method or AP-PCR method is applied to a microorganism group composed of a plurality of microorganisms, there are so many types of DNA fragments to be amplified that it becomes difficult to make the correspondence between the amplified DNA fragments and the microorganism formed as a template, and it is thus difficult to grasp an ecological system formed by the microorganism group. Moreover, it is impossible to discriminate the microorganisms constituting the microorganism group from the amplified DNA fragments.

### DISCLOSURE OF INVENTION

An object of the present invention is to provide a microorganism discriminating method, a microorganism discriminating apparatus, a method of creating database for microorganism discrimination, a microorganism discriminating program and a recording medium on which such a program is recorded, in which apparatus and method, a plurality of microorganisms constituting a microorganism group can simultaneously and easily be discriminated.

A microorganism discriminating method according to one aspect of the present invention is a method of discriminating a target microorganism to be discriminated, including the steps of: preparing a plurality of primers having different amplification probabilities, then with employment of each of the plurality of primers, applying at a time a polymerase chain reaction method in which a thermal denaturation step, a primer annealing step and an extension reaction step with polymerase are repeated in this order, to DNA of the target microorganism to be discriminated, thereby amplifying DNA fragments of the DNA of the target microorganism to be discriminated; applying electrophoresis to the amplified DNA fragments by employing each of the plurality of primers to obtain an electrophoretic image corresponding to each primer; converting the electrophoretic image corresponding to each primer into image data; detecting a band of the electrophoretic image corresponding to each primer on the basis of the image data; finding information as to the position and the intensity of the detected band in the electrophoretic image corresponding to each primer on the basis of the image data; finding a correspondence between the plurality of primers and the information as to the position and the intensity of the band; searching database in which a correspondence with respect to a microorganism to be collated is stored previously, to find a correlation between the correspondence found with respect to the target microorganism to be discriminated and the correspondence found with respect to the microorganism to be collated; and discriminating the target microorganism to be discriminated on the basis of the found correlation.

The microorganism discriminating method in accordance with the present invention enables discrimination and also identification of microorganisms in both cases where a single microorganism is employed as a target microorganism to be discriminated and where a microorganism group composed of a plurality of microorganisms is employed as the target to be discriminated.

When the microorganism group is employed as the target to be discriminated, in particular, it becomes possible to simultaneously discriminate the plurality of microorganisms constituting the microorganism group and further identify them. This makes it possible to clarify the number and the name of the types of microorganisms constituting the microorganism group.

On the other hand, when a single microorganism is employed as the target microorganism to be discriminated, it becomes possible to detect a polymorphism of the target microorganism to be discriminated and a microorganism to be collated with in the database. Also, it becomes possible to find a microorganism that is similar to the target microorganism to be discriminated.

In this microorganism discriminating method, since with employment of each of a plurality of primers having different amplification probabilities, the polymerase chain reaction method is applied at a time to DNA of the target microorganism to be discriminated, and DNA fragments of the target microorganism are amplified to undergo a DNA analysis, such a step of isolating and culturing microorganisms as in a biochemical examination is no longer required. This facilitates the discrimination and identification of microorganisms and also enables the discrimination and identification of such a microorganism that is hard to be isolated. In addition, since the above DNA amplification method employing the plurality of primers is applicable to such a microorganism to be discriminated that has an unknown base sequence, the discrimination and identification of the target microorganism can be carried out without any measurement of its base sequence.

Further, in the above microorganism discriminating method, the polymerase chain reaction method is carried out employing the plurality of primers, and the discrimination is made employing the plural results of the polymerase chain reaction. Thus, even if the individual polymerase chain reaction does not proceed satisfactorily because of various conditions, the individual reaction does not affect to a large extent. This enables a stable and excellent discrimination.

The information as to the position of a band may be expressed by the distance between a predetermined reference position and the band on the electrophoretic image. Alternatively, the information as to the position of the band may be expressed by the size of DNA fragments included in the band.

The information as to the intensity of the band expresses the height or area of a peak in luminous intensity distribution of the band on the electrophoretic image, and such information may be calculated based on gradient distribution of image data.

As for the correspondence between the plurality of primers and the information as to the band position and intensity, the correspondence between information as to the position of each band and information as to the intensity of each band may be indicated for each primer.

A correlation with respect to the target microorganism to be discriminated and the microorganism to be collated with may express a correlation in the intensity of the bands on a substantially identical position with respect to those microorganisms.

The step of discriminating the target microorganism to be discriminated on the basis of the correlation may include the step of calculating a correlation coefficient in the correlation to determine whether or not the target microorganism is the microorganism to be collated with on the basis of whether the calculated correlation coefficient is larger than a predetermined threshold.

The microorganism discriminating method may further include the steps of: applying a polymerase chain reaction method, with employment of a reference primer having a known base sequence, to reference DNA having a base sequence that is complementary with the base sequence of the reference primer, thereby amplifying DNA fragments of the reference DNA; applying electrophoresis to the DNA fragments of the reference DNA amplified by employing the reference primer, to obtain an electrophoretic image corresponding to the reference DNA; converting the electrophoretic image corresponding to the reference DNA into image data; and correcting image data corresponding to the target microorganism to be discriminated on the basis of the image data corresponding to the reference DNA.

In this case, the DNA fragments are securely amplified from the reference DNA by the polymerase chain reaction method employing the reference primer. On the basis of the image data corresponding to the electrophoretic image of the amplified DNA fragments of the reference primer, it becomes possible to evaluate an amplification efficiency of the DNA fragments of the reference DNA in the polymerase chain reaction. The amplification efficiency thus evaluated is also applicable to DNA fragments of the target microorganism to be discriminated. This makes it possible to correct image data corresponding to an electrophoretic image of the DNA fragments of the target microorganism to be discriminated on the basis of the evaluated amplification efficiency and analyze the amount of DNA fragments.

The microorganism discriminating method may further include the steps of: obtaining an electrophoretic image of a DNA size marker simultaneously with the step of obtaining the electrophoretic image corresponding to each primer; converting the electrophoretic image of the DNA size marker into image data; and correcting image data corresponding to the target microorganism to be discriminated on the basis of the image data corresponding to the DNA size marker.

In that case, it is made possible to make accurate comparison between luminous intensities of bands in the electrophoretic images by correcting the gradation of the image data corresponding to the target microorganism on the basis of the image data corresponding to the electrophoretic image of the DNA size marker.

The step of detecting the band of the electrophoretic image may include the step of setting a threshold based on the luminous intensity of the band of DNA fragments amplified from the reference DNA or DNA size marker in the electrophoretic image, to select a band having a luminous intensity that is not less than the threshold in the electrophoretic image.

In this case, a band with a luminous intensity less than the threshold is the band of DNA fragments with lower amplification efficiency and lower reproducibility. On the other hand, a band with a luminous intensity not less than the threshold is the band of DNA fragments with higher amplification efficiency and higher reproducibility. Therefore, it is made possible to analyze only the DNA fragments with higher amplification efficiency and higher reproducibility by selecting the band with the luminous intensity of not less than the threshold. This improves the reliability of the results of discrimination.

The correspondence with respect to plural types of microorganisms may be stored in database. This enables discrimination of various target microorganisms to be discriminated.

A microorganism discriminating apparatus according to another aspect of the present invention is a microorganism discriminating apparatus that discriminates a target microorganism to be discriminated, including: first amplifying means for applying at a time a polymerase chain reaction method in which a thermal denaturation step, a primer annealing step and an extension reaction step with polymerase are repeated in this order, to DNA of the target microorganism to be discriminated, with employment of each of a plurality of primers having different amplification probabilities, thereby amplifying DNA fragments of DNA of the target microorganism to be discriminated; first electrophoresis means for applying electrophoresis to the DNA fragments amplified by the first amplifying means employing each of the plurality of primers, so as to obtain an electrophoretic image corresponding to each primer; first image data converting means for converting the electrophoretic image corresponding to each primer, obtained by the first electrophoresis means, into image data; band detecting means for detecting a band of the electrophoretic image corresponding to each primer on the basis of the image data obtained by the first image data converting means; band information detecting means for finding information as to the position and the intensity of the band detected in the electrophoretic image corresponding to each primer on the basis of the image data; correspondence creating means for creating a correspondence between the plurality of primers and the information as to the band position and the band intensity detected by the band information detecting means; correlation creating means for searching database in which the correspondence with respect to a microorganism to be collated with is stored previously, to create a correlation between the correspondence created with respect to the target microorganism to be discriminated and the correspondence with respect to the microorganism to be collated with; and discriminating means for discriminating the target microorganism on the basis of the correlation created by the correlation creating means.

The microorganism discriminating apparatus according to the present invention is capable of easily carrying out the above-described microorganism discriminating method. Thus, discrimination and also identification of microorganisms can be made in both cases where a single microorganism is employed as a target to be discriminated and a microorganism group composed of a plurality of microorganisms is employed as the target.

When the microorganism group is employed as the target to be discriminated, in particular, it is possible to simultaneously discriminate and identify the plurality of microorganisms constituting the microorganism group. This makes it possible to clarify the number and the name of the types of those microorganisms constituting the microorganism group.

On the other hand, when the single microorganism is employed as the target to be discriminated, it also becomes possible to detect a polymorphism of the target microorganism to be discriminated and a microorganism in database to be collated with. Further, it is possible to find out a microorganism similar to the target microorganism to be discriminated.

In this microorganism discriminating apparatus, since with employment of each of a plurality of primers having different amplification probabilities, the polymerase chain reaction method is applied at a time to DNA of the target microorganism to be discriminated, and DNA fragments of the target microorganism are amplified to undergo a DNA analysis, such a step of isolating and culturing microorganisms as in a biochemical examination is no longer required. This facilitates the discrimination and identification of microorganisms and also enables the discrimination and identification of such a microorganism that is hard to be isolated. In addition, since the above DNA amplification method employing the plurality of primers is applicable to such a microorganism to be discriminated that has an unknown base sequence, the discrimination and identification of the target microorganism can be carried out without any measurement of its base sequence.

Further, in the above microorganism discriminating apparatus, the polymerase chain reaction method is carried out employing the plurality of primers, and the discrimination is made employing the plural results of the polymerase chain reaction. Thus, even if the individual polymerase chain reaction does not proceed satisfactorily because of various conditions, the individual reaction does not affect to a large extent. This enables a stable and excellent discrimination.

The microorganism discriminating apparatus may further include: second amplifying means, with employment of a reference primer having a known base sequence, for applying the polymerase chain reaction method to reference DNA having a base sequence complementary with the base sequence of the reference primer, thereby amplifying DNA fragments of the reference DNA; second electrophoresis means for applying electrophoresis to the DNA fragments of the reference DNA amplified by the reference primer, to obtain an electrophoretic image corresponding to the reference DNA; second image data converting means for converting the electrophoretic image corresponding to the reference DNA into image data; and first correcting means for correcting image data corresponding to the target microorganism to be discriminated on the basis of the image data corresponding to the reference DNA.

In this case, the DNA fragments are securely amplified from the reference DNA by the polymerase chain reaction method employing the reference primer. On the basis of the image data corresponding to the electrophoretic image of the DNA fragments of the reference DNA thus amplified, it becomes possible to evaluate an amplification efficiency of the DNA fragments of the reference DNA in the polymerase chain reaction. The amplification efficiency thus evaluated is applicable also to the DNA fragments of the target microorganism to be discriminated. Accordingly, it becomes possible to correct the image data corresponding to the electrophoretic image of the DNA fragments of the target microorganism on the basis of the evaluated amplification efficiency and analyze the amount of the DNA fragments.

The microorganism discriminating apparatus may further include: third electrophoresis means for obtaining an electrophoretic image of a DNA size marker simultaneously with obtaining the electrophoretic image corresponding to each primer; third image data converting means for converting the electrophoretic image of the DNA size marker into image data; and second correcting means for correcting the image data corresponding to the target microorganism to be discriminated on the basis of the image data corresponding to the DNA size marker.

In this case, accurate comparison in luminous intensities of bands in electrophoretic images is enabled by correcting gradients of the image data corresponding to the target microorganism to be discriminated on the basis of the image data corresponding to the electrophoretic image of the DNA size marker.

The band detecting means may include band selecting means for setting a threshold based on a luminous intensity of the band of the DNA fragments amplified from the reference DNA in the electrophoretic image or the band of the DNA size marker, to select a band having a luminous intensity not less than the threshold in the electrophoretic image.

In this case, a band with a luminous intensity less than the threshold is the band of DNA fragments with lower amplification efficiency and lower reproducibility. On the other hand, a band with a luminous intensity not less than the threshold is the band of DNA fragments with higher amplification efficiency and higher reproducibility. Therefore, it is made possible to analyze only the DNA fragments with higher amplification efficiency and higher reproducibility by selecting the band with the luminous intensity of not less than the threshold. This improves the reliability of the results of discrimination.

The correspondence with respect to plural types of microorganisms may be stored in database. This enables discrimination of various target microorganisms to be discriminated.

A recording medium readable by a computer according to still another aspect of the present invention is a recording medium readable by a computer, on which a microorganism discriminating program for discriminating a target microorganism to be discriminated is recorded, wherein the microorganism discriminating program makes the computer execute the processings of: applying at a time a polymerase chain reaction method in which a thermal denaturation step, a primer annealing step and an extension reaction step with polymerase are repeated in this order, to DNA of the target microorganism to be discriminated, with employment of each of a plurality of primers with different amplification probabilities, thereby amplifying DNA fragments of the DNA of the target microorganism to be discriminated and applying electrophoresis to the DNA fragments amplified by employing each of the plurality of primers, so as to capture a resultant electrophoretic image as image data; detecting a band of the electrophoretic image corresponding to each primer on the basis of the image data; seeking information as to the position and the intensity of the band detected in the electrophoretic image corresponding to each primer on the basis of the image data; finding a correspondence between the plurality of primers and the information as to the band position and the band intensity; searching database in which the correspondence with respect to a microorganism to be collated with is stored previously, to find a correlation between the correspondence found with respect to the target microorganism to be discriminated and the correspondence with respect to the microorganism to be collated with; and discriminating the target microorganism on the basis of the found correlation.

The above-described microorganism discriminating method can easily be carried out in accordance with the recording medium, on which the microorganism discriminating program according to the present invention is recorded.

A method of creating database for microorganism discrimination according to a further aspect of the present invention is a method of creating database in which data of a microorganism to be collated with is stored in order to discriminate a microorganism, including the steps of: preparing a plurality of primers having different amplification probabilities and, with employment of each of the plurality of primers, applying at a time a polymerase chain reaction method in which a thermal denaturation step, a primer annealing step and an extension reaction step with polymerase are repeated in this order, to DNA of a microorganism, thereby amplifying DNA fragments of the DNA of the microorganism; applying electrophoresis to the DNA fragments amplified by employing each of the plurality of primers, to obtain an electrophoretic image corresponding to each primer; converting the electrophoretic image corresponding to each primer into image data; detecting a band of the electrophoretic image corresponding to each primer on the basis of the image data; finding information as to the position and the intensity of the band detected in the electrophoretic image corresponding to each primer on the basis of the image data; finding a correspondence between the plurality of primers and the information as to the band position and the band intensity; and storing in storing means the correspondence as data of the microorganism to be collated with.

In accordance with the database creating method according to the present invention, it is possible to create database for use in the above microorganism discriminating method and apparatus.

A recording medium on which database is recorded according to a further aspect of the present invention is a recording medium on which database storing therein data of a microorganism to be collated with in order to discriminate a microorganism is recorded, wherein the database is created by the method including the steps of: preparing a plurality of primers having different amplification probabilities and, with employment of each of the plurality of primers, applying at a time a polymerase chain reaction method in which a thermal denaturation step, a primer annealing step and an extension reaction step with polymerase are repeated in this order, to DNA of a microorganism, thereby amplifying DNA fragments of the DNA of the microorganism; applying electrophoresis to the DNA fragments amplified by employing each of the plurality of primers, to obtain an electrophoretic image corresponding to each primer; converting the electrophoretic image corresponding to each primer into image data; detecting a band of the electrophoretic image corresponding to each primer on the basis of the image data; finding information as to the position and the intensity of the band detected in the electrophoretic image corresponding to each primer on the basis of the image data; finding a correspondence between the plurality of primers and the information as to the band position and the band intensity; and storing in storing means the correspondence as data of the microorganism to be collated with.

The recording medium recording the database therein according to the present invention can be employed in the above microorganism discriminating method and apparatus.

A microorganism discriminating program according to a further aspect of the present invention is a microorganism discriminating program readable by a computer, which discriminates a target microorganism to be discriminated, and makes the computer execute the processings of: applying at a time a polymerase chain reaction method in which a thermal denaturation step, a primer annealing step and an extension reaction step with polymerase are repeated in this order, to DNA of the target microorganism to be discriminated, with employment of each of a plurality of primers with different amplification probabilities, thereby amplifying DNA fragments of the DNA of the target microorganism to be discriminated and applying electrophoresis to the DNA fragments amplified by employing each of the plurality of primers, so as to capture a resultant electrophoretic image as image data; detecting a band of the electrophoretic image corresponding to each primer on the basis of the image data; finding information as to the position and the intensity of the band detected in the electrophoretic image corresponding to each primer on the basis of the image data; finding a correspondence between the plurality of primers and the information as to the band position and the band intensity; searching database in which the correspondence with respect to a microorganism to be collated with is stored previously, to find a correlation between the correspondence found with respect to the target microorganism to be discriminated and the correspondence with respect to the microorganism to be collated with; and discriminating the target microorganism on the basis of the found correlation.

### BRIEF DESCRIPTION OF THE DRAWINGS

FIG. 1 is a schematic diagram showing an example of a microorganism discriminating apparatus in accordance with the present invention.

FIG. 2 is a flowchart showing an example of a microorganism discriminating method in accordance with the present invention.

FIG. 3 is a flowchart showing an example of an SSC-PCR method for use in the microorganism discriminating method of FIG. 2.

FIG. 4 is a schematic diagram showing an example of a DNA fragment amplifying apparatus for use in the SSC-PCR method of FIG. 3.

FIG. 5 is a schematic diagram showing a process in a first cycle in the SSC-PCR method.

FIG. 6 is a schematic diagram showing a process in a first time of a second cycle in the SSC-PCR method.

FIG. 7 is a schematic diagram showing a process in a second time of the second cycle in the SSC-PCR method.

FIG. 8 is a flowchart showing an example of an image processing method for use in the microorganism discriminating method of FIG. 2.

FIG. 9 is image data of an electrophoretic image processed by using the image processing method of FIG. 8.

FIG. 10 is a diagram showing a method of searching for bands on the same position and with the same primer in band data of a sample microorganism and in band data in database.

FIG. 11 is a diagram showing a correlation between a band intensity of the sample microorganism and that of the database microorganism.

FIG. 12 is a diagram showing a method of calculating correlation coefficients.

FIG. 13 is a block diagram showing the structure of a personal computer for use as an analyzing computer of FIG. 1.

FIG. 14 is a diagram showing a correlation between a band intensity of a sample bacterium No. 2001 and that of a bacterium No. 2001 in database in an Example.

### BEST MODE OF CARRYING OUT THE INVENTION

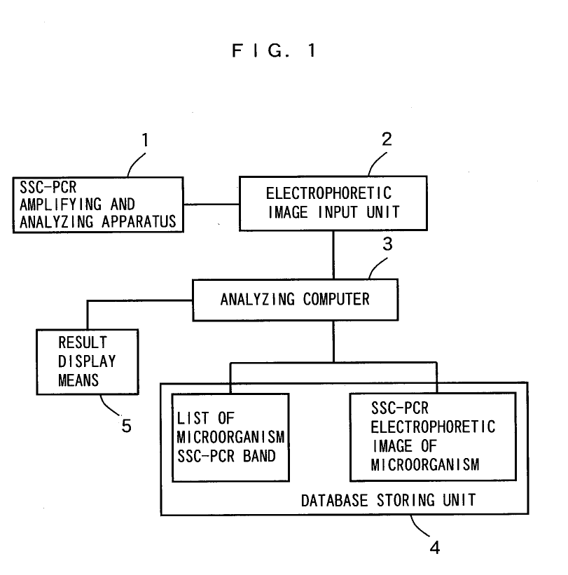

FIG. 1 is a diagram showing a microorganism discriminating apparatus in accordance with the present invention.

With reference to FIG. 1, the microorganism discriminating apparatus includes an SSC-PCR amplifying and analyzing apparatus 1, an electrophoretic image input unit 2, an analyzing computer 3, a database storing unit 4 and result display means 5.

Description will be made later on the SSC-PCR amplifying and analyzing apparatus 1. The electrophoretic image input unit 2 is constituted by a CCD camera and a scanner, for example. The analyzing computer 3 is constituted by a personal computer having such a structure as will be described later. The database storing unit 4 is constituted by a hard disk device and the like, and the result display means 5 is constituted by a display and the like.

The SSC-PCR represents a single strain counting polymerase chain reaction that DNA fragments are amplified on a chain reaction basis from a group of microorganisms or a single microorganism having unknown base sequences by employing a plurality of primers having specific base sequences. Detailed description will be made on the SSC-PCR later.

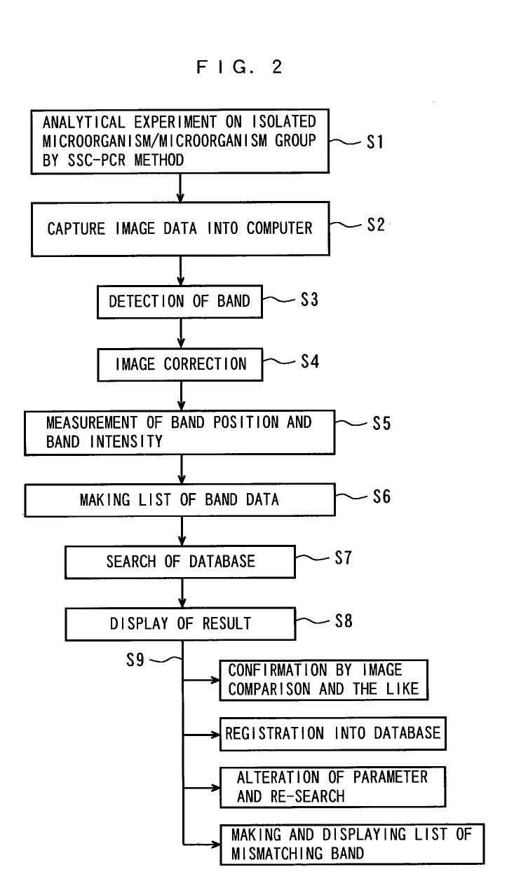

FIG. 2 is a flowchart showing an example of a microorganism discriminating method using the microorganism discriminating apparatus of FIG. 1.

First, as shown in FIG. 2, an analytical experiment is carried out on microorganisms by the SSC-PCR method which will be described later, employing the SSC-PCR amplifying and analyzing apparatus 1 of FIG. 1 (step S1). In this analytical experiment, a single isolated microorganism may be used as a sample, and also, a microorganism group including a plurality of microorganisms may be used as samples.

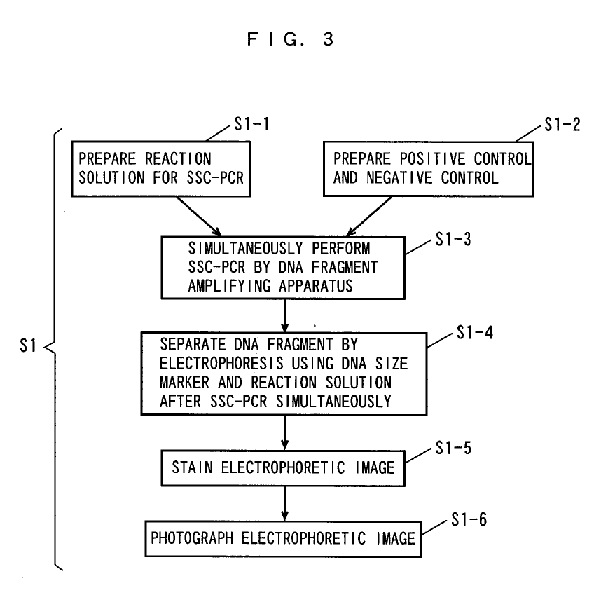

FIG. 3 shows the detail of the analytical experiment on microorganisms by the SSC-PCR method. First, as shown in FIG. 3, predetermined quantities of DNA of a microorganism, a buffer solution for polymerase chain reaction, primers, heat-resistant thermophile DNA polymerase, MgCl₂ and four types of 5′-deoxyribonucleotide triphosphates (dATP, dGTP, dCTP, dTTP) serving as substrates are mixed with each other to prepare a reaction solution for SSC-PCR (step S1-1).

When a microorganism group including a plurality of microorganisms is employed as a sample, DNAs of the plurality of microorganisms are mixed to prepare a reaction solution for SSC-PCR.

The DNA polymerase is an enzyme containing four types of 5′-deoxyribonucleotide triphosphates as substrates for catalyzing a polymerization reaction of DNA strands having base sequences complementary with template DNA. The directionality of polymerization of the DNA strands by the DNA polymerase is at the 5′ to 3′ ends. The primers are DNA fragments (short oligonucleotides) that have 3′-OH groups on their ends, which are essential for the action of the DNA polymerase. Primers each having a specific base sequence and a specific base length are employed in the present invention.

In the SSC-PCR method, as will be described later, since a plurality of primers having different amplification probabilities are employed, a reaction solution for SSC-PCR is prepared for each primer. In this case, for example, since forty-six types of primers are employed, forty-six types of reaction solutions for SSC-PCR containing those different primers are prepared. All of the forty-six types of SSC-PCR reaction solutions are prepared at the same time.

A positive control and a negative control are prepared simultaneously with the preparation of the above-described reaction solutions for SSC-PCR (step S1-2).

The positive control is a sample for use in a control experiment for eliminating errors caused in a series of steps of amplification reaction of DNA fragments which will be described later. In general, it is considered that the amplification efficiency in amplification reaction of DNA fragments is affected by errors caused in a series of steps, for example, errors in concentrations of DNA polymerase, magnesium and the like at preparation of reaction solutions for SSC-PCR, the degree of activation of an employed reagent, errors in temperature at amplification of DNA fragments, and the like. In order to eliminate such errors, such a reaction solution is prepared, as the positive control, that contains a primer amplifying a known type of DNA fragments which are quantitatively analyzable and template DNA having a base sequence complementary with that primer. The DNA fragments amplified in the positive control are quantitatively analyzable by electrophoresis. This makes it possible to evaluate the amplification efficiency of DNA fragments from the quantity of the DNA fragments amplified in the positive control. It is made possible to eliminate the influence exerted by the errors caused in the series of steps of amplification reaction of the DNA fragments by correcting the quantity of DNA fragments in forty-six types of reaction solutions for SSC-PCR on the basis of the amplification efficiency evaluated as above. This enables a comparison in quantity of the amplified DNA fragments.

On the other hand, the negative control is a sample for use in a control experiment for confirming that the amplified DNA fragments belong to a target microorganism group or a target microorganism to be analyzed. In general, microorganisms such as bacteria exist in various places in the air or the like. Thus, it is possible that microorganisms are introduced into the reaction solutions for SSC-PCR in preparation of the reaction solutions. If some microorganism is introduced into any reaction solution, then it becomes impossible to determine whether the amplified DNA fragments are derived from the target microorganism group to be analyzed or from the introduced microorganism. Therefore, as the negative control, a reaction solution for SSC-PCR is prepared that contains a primer but not template DNA. The SSC-PCR and electrophoresis are carried out by employing such a negative control to confirm that no band appears in the electrophoretic image of the negative control. This makes it possible to confirm that the amplified DNA fragments are derived from the target microorganism group or target microorganism to be analyzed.

The primers for use in the positive control and the negative control may have the same base sequences as those of one or two of forty-six types of primers.

After the preparation of the reaction solutions for SSC-PCR as described above, the amplification reaction of the DNA fragments is carried out by the SSC-PCR amplifying and analyzing apparatus 1 (step S1-3).

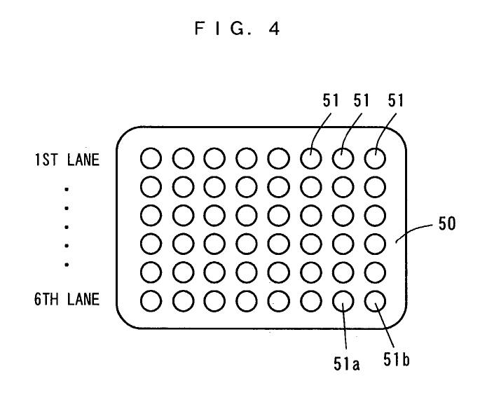

FIG. 4 is a schematic diagram showing an example of a DNA fragment amplifying device in the SSC-PCR amplifying and analyzing apparatus 1. The DNA fragment amplifying device of FIG. 4 is composed of a support plate 50 called a titer plate.

A plurality of openings 51 are formed in an upper surface of the support plate 50. In this case, the forty-six types of reaction solutions for SSC-PCR containing the above-described different amplification probabilities, respectively, are provided successively in the order of the amplification probability of the primers in the plurality of openings 51. Further, the above-described negative control is provided in an opening 51a, while the above-described positive control is provided in an opening 51b.

The amplification of DNA fragments by a SSC-PCR method as described below is carried out at the same time in the forty-six types of reaction solutions for SSC-PCR, the positive control and the negative control by the DNA fragment amplifying device.

In the SSC-PCR method, the following three stage-steps are repeated as in the conventional PCR method. In the SSC-PCR method, however, an analyzable quantity of DNA fragments are amplified by employing primers having specific base sequences for a microorganism or a microorganism group having unknown base sequences.

(1) Thermal Denaturation Step

DNA (initial state) or a DNA fragment is heated and denatured into single strands (DNA strands).

(2) Primer Annealing Step (Primer Bonding Step)

Heat treatment is carried out to bond a primer to an end of an amplification region of a DNA strand.

(3) Extension Reaction Step with Polymerase (Duplication Step with Polymerase)

Starting from the primer, a complementary strand is synthesized by polymerase to form a double strand.

The cycle formed of the above steps (1) to (3) is repeated as one cycle. As a first cycle, the following steps are carried out in the following order: the thermal denaturation step of keeping the above reaction solutions at 94° C. for two minutes, for example; the primer annealing step of keeping the above reaction solutions at 45° C. for two minutes, for example; and the extension reaction step with polymerase of keeping the above reaction solutions at 72° C. for three minutes, for example. The thermal denaturation step in this cycle is set longer than those in the following cycles in order to completely separate long complete DNA into single strands.

As a subsequent second cycle, the following steps are repeated 33 times, for example, in the following order: the thermal denaturation step of keeping the above reaction solutions at 94° C. for one minute, for example; the primer annealing step of keeping the above reaction solutions at 45° C. for two minutes, for example; and the extension reaction step with polymerase of keeping the above reaction solutions at 72° C. for three minutes, for example.

Finally, as a third cycle, the following steps are carried out in the following order: the thermal denaturation step of keeping the above reaction solutions at 94° C. for one minute, for example; the primer annealing step of keeping the above reaction solutions at 45° C. for two minutes, for example; and the extension reaction step with polymerase of keeping the above reaction solutions at 72° C. for ten minutes. The extension reaction step with polymerase in this cycle is set longer than those in the first and second cycles in order to finally complete duplication.

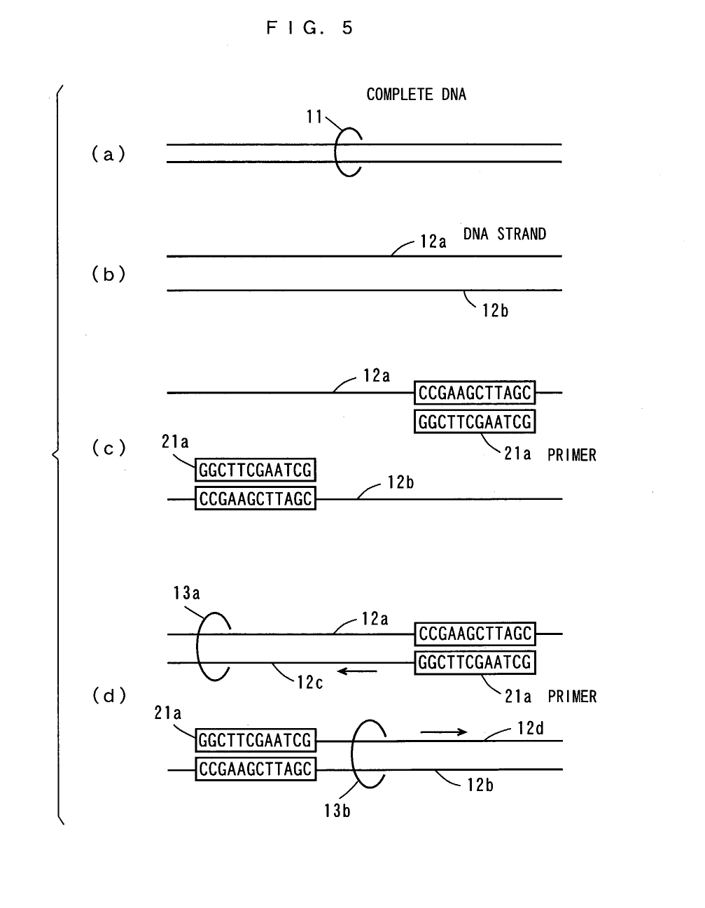

Description will now be made on the first cycle, a first time of the second cycle and a second time of the second cycle with reference to FIGS. 5 to 7. FIGS. 5 to 7 are schematically showing the base sequences of some parts of single strands of DNA and the like, which parts are bonded to primers.

With reference to FIGS. 5 to 7, a primer having a base sequence of GGCTTCGAATCG (sequence No. 14) is employed where T stands for thymine, A for adenine, G for guanine and C for cytosine.

First, as shown in FIG. 5(a), long DNA (complete DNA) 11 contained in a plurality of different microorganisms exists in the above reaction solutions. Description will now be directed to single complete DNA.

First, as shown in FIG. 5(b), in the first cycle, the above long DNA 1 is subjected to the thermal denaturation step longer than those in the second and third cycles, so that the long DNA 1 is heated and denatured, and its double strand is separated from each other into two single strands (DNA strands) 12a and 12b.

Then, as shown in FIG. 5(c), in the primer annealing step, the primer 21a is bonded to be arranged (complementarily arranged) on a compatible position of each of the single strands 12a and 12b compatible with its base sequence. The compatible position is the position of the base sequence to be bonded as viewed from the base sequence of the primer, or the position of a similar base sequence to the base sequence to be bonded as viewed from the base sequence of the primer. In the above SSC-PCR method, the primer is bonded also to a part of the DNA strand having a similar base sequence to its base sequence by setting a lower annealing temperature in the primer annealing step. That is, the primer can be bonded not only to the position of the single strand having the base sequence that is completely complementary with its base sequence but also to the single strand with slight mismatching. FIGS. 5 to 7 illustrate for simplification the case where each primer is bonded to the position of the base sequence to be bonded as viewed from its base sequence.

Then, as shown in FIG. 5(d), an extension reaction is caused by polymerase in the extension reaction step with polymerase, so that single strands 12c and 12d extend along the single strands 12a and 12b, respectively, to form double strands 13a and 13b.

While the strands 13a and 13b that were doubled in the first cycle are separated into the single strands (DNA strands) 12a and 12c and the single strands (DNA strands) 12b and 12d, respectively, thorugh the thermal denaturation step in the first time of the second cycle, a description will now be directed to the single strand 12c separated form the strand 13a.

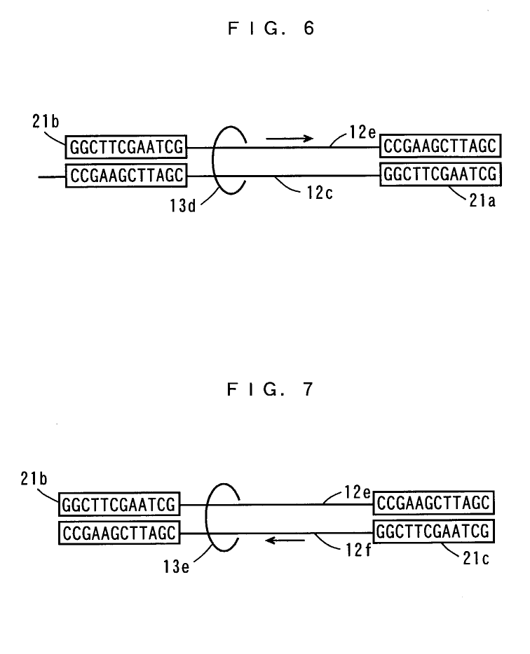

With reference to FIG. 6, a primer 21b is bonded to the single strand 12c separated through the thermal denaturation step so that the primer is arranged on a compatible position in the subsequent primer annealing step. After that, the extension reaction is caused by polymerase in the extension reaction step with polymerase, so that a single strand 12e extends along the single strand 12c to form a double strand 13d.

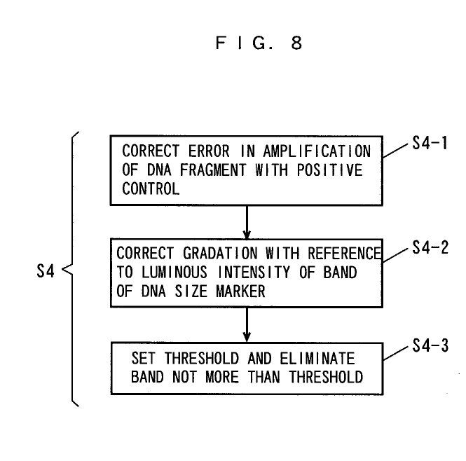

Then, while the double strand 13d is separated into the single strands 12c and 12e through the thermal denaturation step in the second time of the second cycle as shown in FIG. 7, a description will now be directed to the single strand 12e separated from the strand 13d.

With reference to FIG. 7, a primer 21c is similarly bonded to the single strand 12e separated through the thermal denaturation step so that the primer is arranged on a compatible position in the subsequent primer annealing step. After that, the extension reaction is caused by polymerase in the extension reaction step with polymerase, so that a single strand 12f extends along the single strand 12e to form a double strand (DNA fragment) 13e.

The DNA fragment is thus formed, so that another DNA fragment is formed from this DNA fragment, while DNA fragments are formed also from another DNA of the same type followed by continued similar reaction on a chain reaction basis. Thus, the DNA fragments are amplified by this method.

Then, as shown in FIG. 3, by employing the reaction solutions for SSC-PCR, the positive control and the negative control, the DNA fragments amplified in each of the reaction solutions are fractionated size by size (a base pair number) by electrophoresis. At the same time, a quantitatively analyzable DNA size marker with a known concentration is subjected to electrophoresis to be fractionated size by size (step S1-4).

Further, electrophoretic images obtained through the electrophoresis are stained with a fluorochrome (step S1-5), so as to photograph fluorescent images irradiated with ultraviolet rays (step S1-6). A CCD camera, for example, is employed for the photographing. The DNA fragments appear as bands in the electrophoretic images thus obtained.

If no band appears in the electrophoretic image of the negative control, then it is possible to confirm that the amplified DNA fragments are derived from a target microorganism or a target microorganism group to be analyzed.

Then, as shown in FIG. 2, the photographed electrophoretic images are captured as image data into the analyzing computer 3 (FIG. 1) by the scanner of the electrophoretic image input unit 2 (FIG. 1) (step S2), so that each band of the electrophoretic images is detected on the basis of the image data (step S3).

If a primer is strongly bonded to be arranged on a compatible position of template DNA having a predetermined base sequence in the amplification of DNA fragments by the SSC-PCR method, the DNA fragments have high amplification efficiency. The DNA fragments amplified by such a primer appear as a clear band with high reproducibility in the electrophoretic image. On the other hand, if the primer is weakly bonded to the compatible position of the template DNA, the primer is bonded to another position that has stronger bonding than the compatible position of the template DNA. Since the amplification reaction of DNA fragments thus proceeds competitively, the amplification efficiency of the DNA fragments becomes lower in the case of the weak bonding between the primer and the template DNA. The DNA fragments amplified by such a primer with weak bonding appear as an unclear band with low reproducibility in the electrophoretic image. Because of such DNA fragments with low reproducibility included as described above, the data obtained by SSC-PCR method has lower reliability as a whole.

In order to improve the reliability of the data obtained by the SSC-PCR method, image data of the electrophoretic image is subjected to image correction as described below (step S4).

FIG. 8 is a flowchart showing an example of the step of image correction. In the image data, with reference to FIG. 8, the DNA fragments amplified in the positive control are quantitatively analyzed by comparison between a luminous intensity of the band of the positive control and that of the DNA size marker having a known quantity. Further, the amplification efficiency in the DNA fragment amplification reaction is evaluated by using the quantitatively analyzed value. The luminous intensities of the bands of forty-six types of reaction solutions for SSC-PCR are corrected on the basis of the evaluated amplification efficiency (step S4-1). This eliminates any influences exerted on the amplification efficiency of the DNA fragments by errors in reaction conditions and the like in the DNA amplification reaction.

The luminous intensity here indicates the height of peaks in luminous intensity distributions.

Alternatively, as a method of quantitatively analyzing the positive control, another method of measuring absorption of ultraviolet rays at 260 nm after purifying the DNA fragments may be employed.

Moreover, the luminous intensities of the bands of the forty-six types of reaction solutions for SSC-PCR are corrected by using as a reference the luminous intensity of the band of the quantitatively analyzable DNA size marker. Thus, the gradation (gray scale) of the image data of the electrophoretic image is corrected (step S4-2).

In general, errors occur in the gradation (gray scale) of the electrophoretic images depending on the degree of staining with ethidium bromide and the degree of exposure in photographing. However, such errors can be eliminated by correcting the gradation of the image data of the electrophoretic image on the basis of the luminous intensity of the band of the DNA size marker having a known concentration.

Then, a threshold is set on the basis of the luminous intensity of the band of the quantitatively analyzable DNA size marker, for eliminating bands with luminous intensities less than the threshold (step S4-3). This makes it possible to eliminate bands with lower amplification efficiency and lower reproducibility. By analyzing bands with higher amplification efficiency and higher reproducibility thus obtained, highly reliable data is obtained.

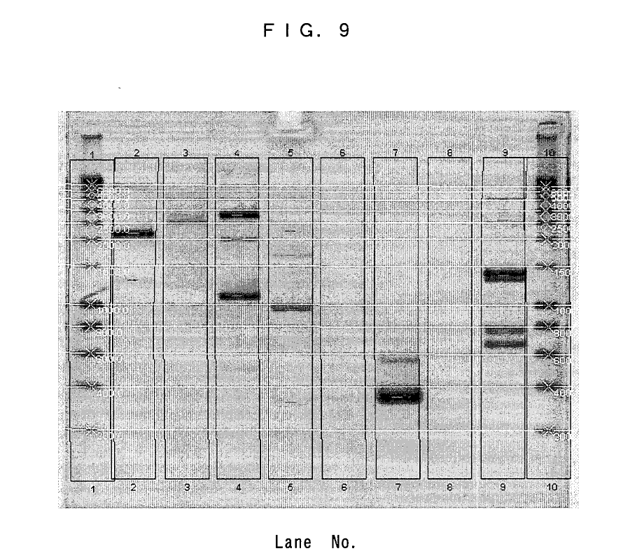

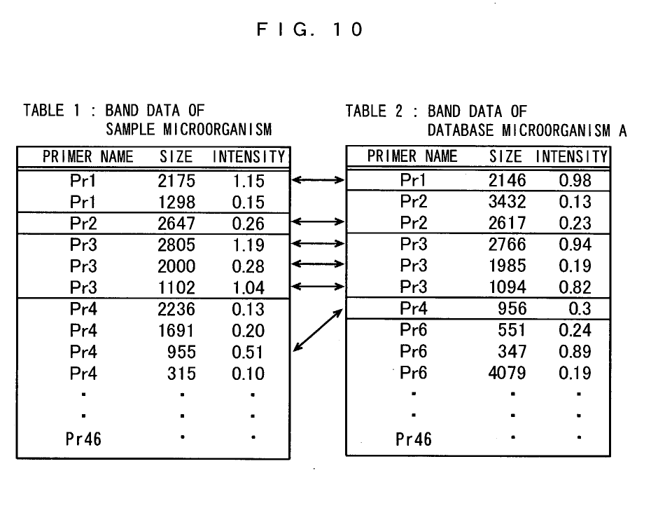

FIG. 9 is a diagram showing an example of image data of electrophoretic images subjected to the image correction as describe above. In FIG. 9, lanes 1 and 10 indicate data of the electrophoretic images of the DNA size marker, and lanes 2 to 9 indicate data of the electrophoretic images in primers of sequence Nos. 1 to 8. Such electrophoretic data as shown in FIG. 9 are obtained also with respect to each of primers of sequence Nos. 9 to 46.

Then, as shown in FIG. 2, by comparing the position of the band in each primer with that in the DNA size marker, the position of the band in each primer is converted into a band size (base pair number) and measured. Further, a band intensity (the luminous intensity of a band) is measured with respect to the band on each position (step S5). The band intensity here means the height of peaks in the luminous intensity distribution of bands.

The measurement of the band position and the band intensity is carried out by the analyzing computer 3 (FIG. 1).

In addition, data of the band position and the band intensity in each primer obtained as above are summed up to make a list of band data, by employing the analyzing computer 3 (step S6).

Normally, in the SSC-PCR method, a plurality of PCR reactions (polymerase chain reaction) are carried out using a plurality of different types of primers. Thus, one electrophoretic image is obtained for each primer as the result of SSC-PCR. Since forty-six types of primers are employed in this case, forty-six types of electrophoretic images are obtained.

In such a case, for example, that the electrophoretic images of eight types of primers are shown in one photograph as in FIG. 9 described above, the electrophoretic images of forty-six types of primers are shown in six photographs in total. Thus, image data is obtained for each photograph.

In the analyzing computer 3 of the microorganism discriminating apparatus of FIG. 1, it is possible to collect the band data of those forty-six types of primers indicated over such a plurality of photographs and sum up the collected data to single data. This makes it possible to make a list of band data of SSC-PCR with respect to a sample microorganism.

Primer names (sequence numbers), band positions represented by size, and band intensities are listed on the list of the band data made by the analyzing computer 3. Table 1 shows the list of the band data.

TABLE 1

| Primer Name | Size | Intensity |
|---|---|---|
| Pr1 | 2175 | 1.15 |
| Pr1 | 1298 | 0.15 |
| Pr2 | 2647 | 0.26 |
| Pr3 | 2805 | 1.19 |
| Pr3 | 2000 | 0.28 |
| Pr3 | 1102 | 1.04 |
| Pr4 | 2236 | 0.13 |
| Pr4 | 1691 | 0.20 |
| Pr4 | 955 | 0.51 |
| Pr4 | 315 | 0.10 |
| . | . | . |
| . | . | . |
| . | . | . |
| Pr46 | | |

In the item of Primer Name in Table 1, a primer of sequence No. 1 is designated as Pr1.

While the foregoing description has been made on the case where the band position in each primer is indicated as the band size, the band position may be indicated not only as the band size but also on the basis of a measured distance as will be described later in the Example. In this case, the ratio of measured distances, for example, is designated in the item of the band position on the list of band data.

While the foregoing description has been made on the case where the height of peaks of the luminous intensity distribution of bands is measured as the band intensity, the area (capacity) of the luminous intensity distribution of bands may be measured as the band intensity. Since the area of the luminous intensity distribution of bands corresponds to the content of DNA contained in bands, it is more preferable to employ the area of the luminous intensity distribution of bands as the band intensity than employing the height of peaks thereof.

In addition, the order of the band detection, the image correction and the measurement of band position and band intensity is not limited to the above order, but sometimes becomes the opposite order if necessary.

Then, as shown in FIG. 2, data of a plurality of microorganisms registered in the database of the database storing unit 4 (FIG. 1) are searched on the basis of the data of the sample microorganism to compare the band data of the sample microorganism with band data with respect to primers Pr1 to Pr46 of the plurality of microorganisms sequence Nos. 1 to 46 in the database, by employing the analyzing computer 3 (FIG. 1). Further, correlation coefficients with respect to the sample microorganism and each of the plurality of microorganisms in the database are evaluated, so that a microorganism corresponding to the sample microorganism is searched from the database on the basis of the evaluated correlation coefficients (step S7).

The detail of the database searching (step S7) will now be described in due order.

\mathring{1} Searching for Band on Identical Position in Identical Primer in Sample Microorganism and Database Microorganism

A list of data of SSC-PCR, for example, image data of electrophoretic images and band data with respect to each of a plurality of microorganisms identified by a biochemical examination and the like is registered in the database of the database storing unit 4 of the microorganism discriminating apparatus, as shown in FIG. 1. The structure of the list of band data registered in the database is the same as that of the sample microorganism band data list made as above (Table 1).

Table 2, for example, shows a list of band data of a microorganism A registered in the database. This microorganism A is a bacterium.

TABLE 2

| Primer Name | Size | Intensity |
|---|---|---|
| Pr1 | 2146 | 0.98 |
| Pr2 | 3432 | 0.13 |
| Pr2 | 2617 | 0.23 |
| Pr3 | 2766 | 0.94 |
| Pr3 | 1985 | 0.19 |
| Pr3 | 1094 | 0.82 |
| Pr4 | 956 | 0.3 |
| Pr6 | 551 | 0.24 |
| Pr6 | 347 | 0.89 |
| Pr6 | 4079 | 0.19 |
| . | . | . |
| . | . | . |
| . | . | . |
| Pr46 | | |

A band on the identical position in the identical primer is searched on the basis of the list of band data of the sample microorganism (Table 1) from the list of band data of the plurality of microorganisms registered in such database.

In this searching, if the position of the band of some microorganism in the database is shifted within a certain range from the position of the band of the sample microorganism, those bands are regarded as being on the same position. The range of the band position to be regarded as the same position is determined on the basis of errors in the experiment and is hence changed if necessary.

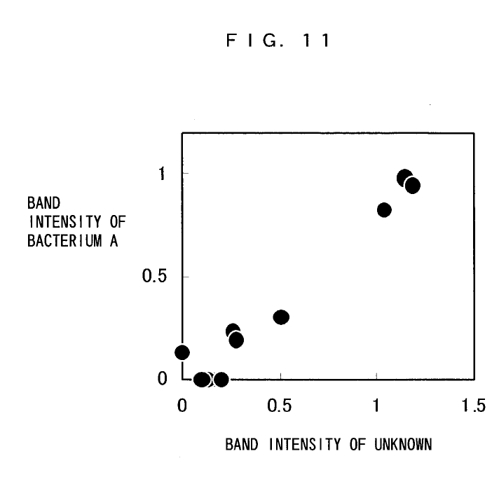

If the band data of the database microorganism A (Table 2) is searched on the basis of the band data of the sample microorganism (Table 1), for example, as shown in FIG. 10, such bands as denoted with the arrows in FIG. 10 are searched as those on the identical positions in the identical primers.

The searching for the bands on the identical positions in the identical primers is carried out also with respect to each of the plurality of other microorganisms than the microorganism A registered in the database.

\mathring{2} Making Table for Comparison between Sample Microorganism Data and Database Microorganism Data

Then, a list is made that displays data of all band positions (sizes) and band intensities in primers Pr1 to Pr46 with respect to the sample microorganism and the database microorganism by collecting the band data of the sample microorganism and those of the database microorganism and summing them up in one data.

Table 3, for example, is a list of band data summing up the data of the sample microorganism (Table 1) and those of the database microorganism A (Table 2).

TABLE 3

Band Intensity

| Primer | | Sample | Database |
|---|---|---|---|
| Name | Size | Microorganism | Microorganism A |
| Pr1 | 2175 | 1.15 | 0.98 |
| Pr1 | 1298 | 0.15 | 0 |
| Pr2 | 3432 | 0 | 0.13 |
| Pr2 | 2647 | 0.26 | 0.23 |
| Pr3 | 2805 | 1.19 | 0.94 |
| Pr3 | 2000 | 0.28 | 0.19 |
| Pr3 | 1102 | 1.04 | 0.82 |
| Pr4 | 2236 | 0.13 | 0 |
| Pr4 | 1691 | 0.20 | 0 |
| Pr4 | 955 | 0.51 | 0.3 |
| Pr4 | 315 | 0.10 | 0 |
| . | . | . | . |
| . | . | . | . |
| . | . | . | . |
| Pr46 | | | |

With reference to Table 3, as for the bands on the identical positions in the identical primers (the bands denoted with the arrows in FIG. 10) in the sample microorganism and the database microorganism A, searched in the above step \mathring{1}, the band sizes of the sample microorganism are designated in the item of Band Size in Table 3.

As for a band that exists in the sample microorganism but not in the database microorganism A such as the band of size 1298 of a primer Pr1, for example, the band intensity of the database microorganism A is assumed to be 0. On the other hand, as for a band that exists in the database microorganism but not in the sample microorganism such as the band of size 3432 of a primer Pr2, for example, the band intensity of the sample microorganism is assumed to be 0.

Such a list of band data as shown in Table 3 is also made for each of the plurality of other microorganisms than the microorganism A registered in the database.

\mathring{3} Calculation of Correlation Coefficients in Sample Microorganism and Database Microorganism

On the basis of the above list of band data (Table 3), comparison is made between the band intensity of the sample microorganism and that of the database microorganism in each band, so as to find out the correlation between those microorganisms.

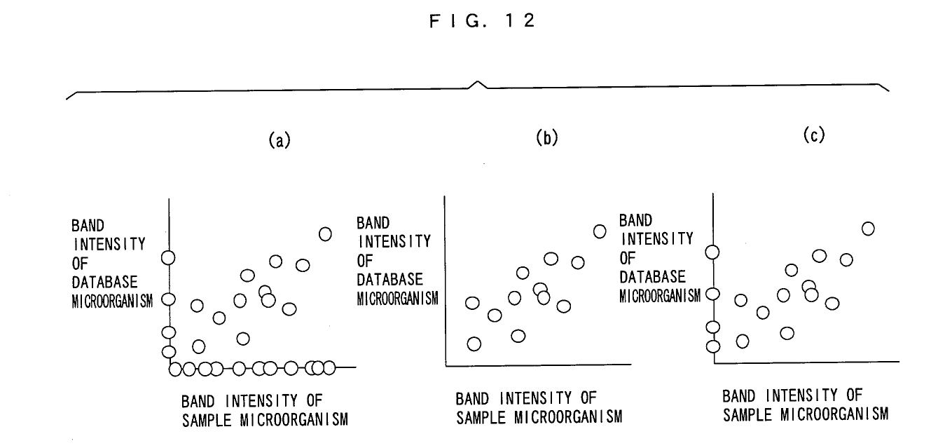

When comparison is made between the band intensity of the sample microorganism and that of the database microorganism A for each band on the basis of the list of band data in the sample microorganism and the database microorganism A, for example, such a correlation as shown in FIG. 11 is obtained. The abscissa represents the band intensity of the sample microorganism, and the ordinate represents the band intensity of the database microorganism A in FIG. 11. While all of the bands are not plotted in FIG. 11, all of the bands are actually plotted for finding out the correlation.

As in the foregoing manner, a correlation coefficient in the sample microorganism and the database microorganism is calculated by comparison between the band intensity of the sample microorganism and that of the database microorganism A.

As for each of the plurality of other microorganisms than the microorganism A registered in the database also, a correlation between each of those microorganisms and the sample microorganism is found out by the same comparison as the above, so as to calculate each correlation coefficient. The calculation of correlation coefficients is carried out by employing the analyzing computer 3 (FIG. 1).

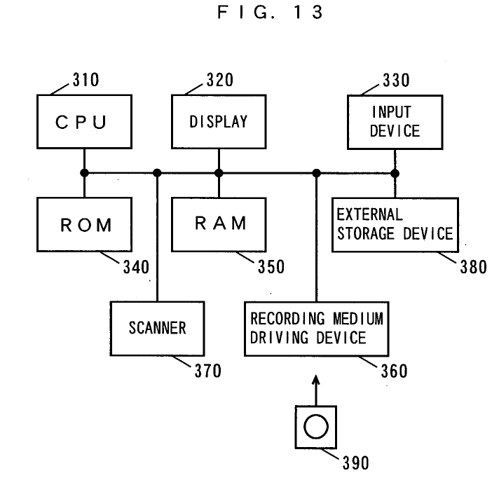

As a method of calculating correlation coefficients, there are three methods shown in FIGS. 12(a) to (c) depending on the way of taking data for use in calculation. These three methods will now be described.

(a) Method of Calculating Correlation Coefficient Employing All Data of Sample Microorganism and Database Microorganism

As shown in FIG. 12(a), according to this method, a correlation coefficient is calculated by using all the data of the respective band intensities of the sample microorganism and the database microorganism including data of 0. In other words, the correlation coefficient is evaluated in view of all plots including the plot on the ordinate and that on the abscissa in the correlation diagram.

If this method is applied in the case where a sample is a mixture of a plurality of microorganisms, a correlation coefficient evaluated by calculation becomes a small value even though there is a correlation between the sample and a predetermined microorganism in the database. Accordingly, the method (a) is preferably applied in the case where a sample is a single microorganism.

(b) Method of Calculating Correlation Coefficient Employing Only Data of Bands Existing in Both Sample Microorganism and Database Microorganism

As shown in FIG. 12(b), according to this method, a correlation coefficient is calculated with respect to the data of the respective band intensities of the sample microorganism and the database microorganism except all the data of 0. That is, the correlation coefficient is calculated in view of other plots than the plot on the ordinate or that on the abscissa in the correlation diagram.

As has been described in the above method (a), if a sample is a mixture of a plurality of microorganisms, it is preferable to exclude data that the band intensity of the database microorganism is 0, in order to obtain a higher correlation coefficient. Further, there is a case where a band that should appear does not actually appear due to some causes in the PCR reaction. In this case, it is preferable to exclude data that the band intensity of the sample microorganism is 0, in order to obtain a higher correlation coefficient.

(c) Method of Calculating Correlation Coefficient Employing All Data of Bands Existing in Database Microorganism

As shown in FIG. 12(c), according to this method, a correlation coefficient is calculated by employing the data of the sample microorganism including data of 0 and the data of the database microorganism not including data of 0. That is, the correlation coefficient is calculated in view of not the plot on the abscissa but other plots including the plot on the ordinate in the correlation diagram.

If a sample is a mixture of a plurality of microorganisms, for example, the correlation coefficient should be calculated by employing all the band data of the sample microorganism including the data of 0, in order to confirm the type of the microorganisms constituting the sample. This is because when only several bands of the database microorganism match the bands of the sample microorganism, and happen to exhibit a good correlation, if the correlation coefficient is calculated by the above method (b) not in view of the 0 data of the sample, then a higher correlation coefficient value than the actual value is evaluated irrespectively of the fact that several bands are in correlation. In contrast, if the method (c) taking into consideration the 0 data of the sample microorganism is employed in such a case, then the correlation coefficient does not become as high as the one evaluated by the method (b).

\mathring{4} Setting Threshold for Searching

After the correlation coefficient between the sample microorganism and each of the plurality of microorganisms registered in the database is calculated as described above, a threshold is set with respect to the evaluated coefficient. On the basis of the set threshold, a microorganism having a correlation coefficient higher than the threshold is selected as a microorganism to be searched.

The threshold is in the range from 0.2 to 0.6, preferably 0.25 to 0.35. As the threshold is set higher, accuracy for searching becomes higher. Conversely, if the threshold is set lower, then it becomes possible to search for possible microorganisms widely from the database.

Finally, as shown in FIG. 2, the names of microorganisms indicating higher values than the threshold are displayed as the result of searching in the order of higher correlation coefficients (step S8).

The following step (step S9) may be provided if necessary.

For example, the step of confirmation by comparison of image data and the like may be provided as shown in FIG. 2. In this step, confirmation is made by the following method wherther the microorganism displayed as the result of searching is identical to the sample microorganism or one of the microorganisms constituting the sample.

For example, the image data of the electrophoretic image of the sample microorganism may be read, while the image data of the electrophoretic image of the searched microorganism may be read from the database storing unit 4, so as to compare those read image data. Further, the correlation state between the sample microorganism and the searched microorganism may be examined by displaying the diagram of correlation between the respective band intensities thereof, as shown in FIG. 11. Such methods enable the confirmation of the results of searching.

Moreover, the step of registration in the database may be provided. In this step, if data search is carried out employing a single microorganism as a sample, and consequently, it becomes clear that the microorganism identical to the employed sample microorganism does not exist in the database, then data with respect to this sample microorganism is newly registered in the database of the database storing unit 4. This makes a broader range of microorganisms that can be searched.

Further, the step of altering parameters and the like and re-searching may be provided. In this step, alteration is made for, for example, the range of the position of bands regarded as those on the identical position as described in the step \mathring{1} in the database search (step S7), and for setting the threshold for searching as described in the step \mathring{4} in the database search (step S7), so that re-search is carried out. Alternatively, alteration is made for the method of calculating the correlation coefficient as described in the step \mathring{3} in the database search (step S7), for carrying out re-search. This makes it possible to carry out searching suitable for the sample microorganism so as to obtain a result.

In addition, the step of making a list of mismatching bands between the sample microorganism and the searched microorganism and displaying such a list may be provided. In this step, the list of the bands of the sample microorganism that do not match those of the searched microorganism ( i.e., mismatching bands) is created and displayed.

When a single type of microorganism is searched by employing a single microorganism for a sample, it is possible that the mismatching band between the sample microorganism and the searched microorganism may correspond to a polymorphism (a different property) that enables the distinction between the sample microorganism and the searched microorganism. On the other hand, when a mixture of a plurality of microorganisms is employed as a sample, it is highly possible that the mismatching band between the sample microorganism and the searched microorganism may be the band of some microorganism that is not registered in the database 4. Since such a mismatching band can be important information as to the sample microorganism as described above, the mismatching band is preferably left as data.

According to the above-described microorganism discriminating method in accordance with the present invention, it is possible to discriminate and also identify both the single microorganism and the microorganism group constituted by the plurality of microorganisms employed as a sample.

In particular, when the microorganism group is employed as a sample, it is possible to simultaneously discriminate and also identify the plurality of microorganisms constituting the group. Thus, it becomes possible to make clear the number and the name of the types of microorganisms constituting the microorganism group.

On the other hand, when a single microorganism is employed as a sample, it also becomes possible to detect a polymorphism of the sample microorganism and the database microorganism. it is also possible to find out any microorganism similar to the sample microorganism.

In this microorganism discriminating method, since a DNA analysis is made on the sample microorganism (or the microorganism group) by SSC-PCR, the step of isolating and culturing microorganisms such as in a biochemical examination is unnecessary. This enables easy discrimination and easy identification while enabling discrimination and identification of microorganisms that are hard to be isolated. Further, since the SSC-PCR is applicable to such a sample that has an unknown base sequence, it is possible to discriminate and identify sample microorganisms without measuring their base sequences.

Moreover, since the PCR reactions are carried out, respectively, employing a plurality of primers, and analyses are made employing the plural results of the PCR reactions in this microorganism discriminating method, those individual PCR reactions exert less influences on such analyses even if the individual PCR reactions do not proceed satisfactorily due to various conditions. Thus, stable excellent analyses can be made.

For example, it is made possible to identify microorganisms in a tank of a organic waste processor or the soil by applying the above-described microorganism discriminating method to a plurality of microorganisms sampled from the tank or the soil. It is also made possible to carry out excellent organic waste processor and create good compost by changing conditions of the tank of the organic waste processor on the basis of the result of the above identification.

In addition, such a microorganism discriminating method is effective in discovering the soil, food or the like contaminated by toxic contaminants such as mercury, arsenic, dioxin, environmental hormones and the like and knowing the contaminated state of the soil, food or the like. That is, if the microorganisms searched by this method include those related to the contaminants, then such a possibility is suggested that the contaminants are contained in the soil or the like from which the microorganisms have been sampled. Further, the contaminated state is suggested depending on the degree of existence of the microorganisms related to the contaminants.

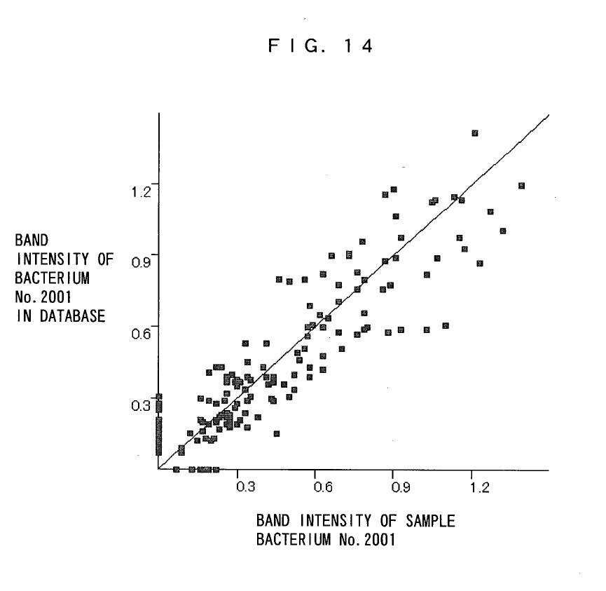

FIG. 13 is a block diagram showing the structure of a personal computer employed as the analyzing computer 3 of FIG. 1.

The personal computer of FIG. 13 includes a CPU (central processing unit) 310, a display 320, an input device 330, a ROM (read-only memory) 340, a RAM (random access memory) 350, a recording medium driving device 360, a scanner 370 and an external storage device 380.

The display 320 is constituted by a liquid crystal display panel, and a CRT (cathode ray tube) and the like and is used as the result display means 5 of FIG. 1. The input device 330 is constituted by a keyboard, a mouse and the like and is used for input of various types of data and instructions. The ROM 340 stores a system program therein.

The recording medium driving device 360 is constituted by a CD-ROM drive, a floppy disk drive and the like and carries out reading and writing of data to a recording medium 390 such as a CD-ROM, a floppy disk and the like. A microorganism discriminating program for carrying out processing in the steps S2 to S8 in the microorganism discriminating method of FIG. 2 is recorded on the recording medium 390.

The scanner 370 inputs as image data an electrophoretic image photographed by the CCD camera and stores the input image data in the external storage device 380. The scanner 370 serves as the electrophoretic image input unit 2 of FIG. 1.

The external storage device 380 is constituted by a hard disk unit and the like and stores the microorganism discriminating program read from the recording medium 390 through the recording medium driving device 360. Further, the external storage device 380 stores the above database therein. The database storing unit 4 of FIG. 1 is constituted by the external storage device 360. The CPU 310 executes the microorganism discriminating program stored in the external storage device 380, on the RAM 350.

As the recording medium 390 recording the microorganism discriminating program, various recording media such as a semiconductor memory such as a ROM, a hard disk the like can be employed. Alternatively, the microorganism discriminating method may be downloaded to the external storage device 360 via a communication medium such as a communication line and the like to be executed on the RAM 350. In this case, the communication medium corresponds to the recording medium.

In the present embodiment, the SSC-PCR amplifying and analyzing apparatus 1 corresponds to first, second and third amplifying means, the electrophoretic image input unit 2 corresponds to first, second and third image data converting means, and the analyzing computer 3 constitutes band detecting means, band information detecting means, correspondence creating means, first and second correcting means, correlation creating means and discriminating means.

The database stored in the database storing unit 4 is created by carrying out processings in the steps S1 to S6 of FIG. 2 with respect to a target microorganism to be collated with.

### EXAMPLE

In the example, a DNA analysis is first made by the SSC-PCR method with respect to each of six types of bacteria isolated from the organic waste processor, and the resultant data is registered in the database.

Then, the DNA analysis is made again by the SSC-PCR method, employing, as samples, bacteria identical to the six types of bacteria employed in the creation of the database. On the basis of analysis data of those samples, the types of the sample bacteria are searched from the above created database, so as to determine whether the sample bacteria match the searched bacteria. A detailed description will now be made.

1. Creation of Database

\mathring{1} Method of Driving Organic Waste Processor

A household organic waste processor SNS-T1 (outer dimensions: 580 by 450 by 795 mm) manufactured by Sanyo Electric Co., Ltd. was employed and improved by connecting an air pump and an air adjuster to an outlet attached to this organic waste processor. This organic waste processor was set in a laboratory with a small temperature variation.

Wood chips (cedar material of 1.5 mm in mean particle diameter) of 25 kg (water content: 70%) were introduced as a treating carrier into a tank of the organic waste processor. Kitchen garbage of 1 kg composed of 450 g of vegetables, 300 g of fruits, 40 g of fish, 30 g of meat and 180 g of cooked rice was introduced into the tank once a day, five times a week, and thereafter the garbage in the tank was stirred with stirring blades of the organic waste processor.

The water content of the wood chips in the tank was adjusted to 35 to 45% to keep an excellent treating state. Fine adjustment of the water content was made by adjusting the amount of airflow from the air pump by the air adjuster.

\mathring{2} Isolation of Bacteria for Treating Kitchen Garbage

Five types of agar culture media for culturing bacteria were prepared. The composition of each agar culture medium for culturing bacteria is shown in Tables 4 to 8 below.

TABLE 4

| | |
|---|---|
| nutrient broth medium(Eiken E-MC35) | 18 g/L |
| Sodium chloride | 0.5 M |
| pH | prepared to 9 with |
| | sodium hydroxide |
| agar | 15 g/L |

TABLE 5

| | |
|---|---|
| nutrient broth medium(Eiken E-MC35) | 18 g/L |
| Sodium chloride | 0.25 M |
| PH | prepared to 5 with 1 N |
| | hydroxide acid |
| agar | 15 g/L |

TABLE 6

| | |
|---|---|
| nutrient broth medium(Eiken E-MC35) | 18 g/L |
| Sodium chloride | 0.25 M |
| pH | prepared to 11 with |
| | sodium hydroxide |
| agar | 15 g/L |

TABLE 7

| | |
|---|---|
| nutrient broth medium(Eiken E-MC35) | 18 g/L |
| Sodium chloride | 2.0 M |
| pH | prepared to 11 with |
| | sodium hydroxide |
| agar | 15 g/L |

TABLE 8

| | |
|---|---|
| nutrient broth medium(Eiken E-MC35) | 18 g/L |
| Sodium chloride | 0.25 M |
| PH | prepared to 7 with |
| | sodium hydroxide |
| agar | 15 g/L |

On the 490th day from starting of driving the organic waste processor, 10 g of the wood chips were sampled from the tank and were suspended until bacteria were sufficiently separated from the wood chips with addition of 90 mL of a sterilized 0.85% saline solution. A resultant suspension was diluted to 10⁻⁶, and 100 μL of the diluted suspension was homogeneously inoculated on each agar medium. After culture at 37° C. for three days, all colonies were transferred to a new agar medium for isolation of the bacteria.

Six different types of bacteria among the isolated colonies were employed as samples for SSC-PCR. Those isolated bacteria were Nos. 1028, 1030, 2001, 4004, 5063 and 7004, respectively; the bacteria Nos. 1028 and 1030 employed the medium with the composition shown in Table 4, the bacterium No. 2001 employed the medium with the composition shown in Table 5, the bacterium No. 4004 employed the medium with the composition shown in Table 6, the bacterium No. 5063 employed the medium with the composition shown in Table 7, and the bacterium No. 7004 employed the medium with the composition shown in Table 8 for each isolation.

Chromosome DNA of each bacterium was prepared according to the method of “Preparation of Genomic DNA from Bacteria” described in Current Protocols in Molecular Biology (published by Greene Publishing Associates and Wiley-Interscience), 2.4.1.-2.4.2.

\mathring{3} DNA Analysis on Six Types of Bacteria Isolated from Organic Waste Processor

An analysis was made on each bacterium by a SSC-PCR method which will be described below, by employing PCR System 9700 of PE Applied Biosystem and DNA amplifier MIR-D40 manufactured by Sanyo Electric Co., Ltd.

Forty-six types of primers (sequence numbers 1 to 46) as shown in Table 9 below were employed in the SSC-PCR method.

TABLE 9

| | Primer | Base Sequence |
|---|---|---|
| No. | Name | 5′→3′ |
| 1 | D27 | AGAATGTCCGTA |
| 2 | A09 | CCGCAGTTAGAT |
| 3 | B03 | CAGTGGGAGTTT |
| 4 | B62 | TCTATGGACCCT |
| 5 | C11 | TTCATTCTGGGG |
| 6 | C23 | CCGTCTTTTCTG |
| 7 | A82 | TGGCCTATTGGC |
| 8 | B02 | GTCATGCCTGGA |
| 9 | A30 | GACCTGCGATCT |
| 10 | A81 | TGGCCTCTTGGA |
| 11 | A83 | GGTTTCCCAGGA |
| 12 | B06 | TCGTCCGGAGAT |
| 13 | C05 | CGCTTCGTAGCA |
| 14 | H81 | GGCTTCGAATCG |
| 15 | D26 | GATGAGCTAAAA |
| 16 | A70 | GAGCAGGAATAT |
| 17 | B30 | CTTAGGTTACGT |
| 18 | D04 | GTGGATCTGAAT |
| 19 | A63 | CCTATCCCAACA |
| 20 | D30 | GAGACTACCGAA |
| 21 | C66 | GACAGCGTCCTA |
| 22 | B09 | CTTGAGCGTATT |
| 23 | B10 | ACTGAGATAGCA |
| 24 | B42 | GAGAGACGATTA |
| 25 | A89 | GACGCCCATTAT |
| 26 | B66 | GACGGTTCTACA |
| 27 | C46 | GATGGTCCGTTT |
| 28 | H83 | TTCACCAACGAG |
| 29 | B07 | CAGGTGTGGGTT |
| 30 | A86 | ATTGGTGCAGAA |
| 31 | A90 | AAGGCGTGTTTA |
| 32 | C30 | TATTGGGATTGG |
| 33 | A92 | AACATCTCCGGG |
| 34 | B01 | ATCATTGGCGAA |
| 35 | B69 | TTGAGTAGTTGC |
| 36 | A91 | TACGCCGGAATA |
| 37 | A67 | CCTGAGGTAGCT |
| 38 | C47 | GCCGCTTCAGCT |
| 39 | B90 | ATCTAAACCACG |
| 40 | C65 | AGAGCTGAAGTA |
| 41 | C09 | GCCTTCGTTACG |
| 42 | C62 | AGGGCTCTAGGC |
| 43 | C82 | TTGCATAATCGT |
| 44 | C08 | GGCAGATATCAT |
| 45 | A42 | TCCAAGCTACCA |
| 46 | B72 | TAACAACCGAGC |

The composition of a reaction solution employed in the PCR method is shown in Table 10.

TABLE 10

| | Final Concentration | |
|---|---|---|
| Buffer | | |
| Tris-HC1 | 10 | mM |
| KC1 | 50 | mM |
| MgCl₂ | 1.5 | mM |
| dNTPmix | 200 | μM |
| Primer | 2 | μM |
| Chromosome DNA of Bacteria | 1 | μg/L |
| Taq DNA Polymerase | 0.025 | unit/μL |

In Table 10, Tris of Tris-HCl is the abbreviation of Tris(hydroxymethyl)aminomethane. dNTPmix stands for an isosbestic mixed solution of dATP(2′-deoxyadenosine-5′-triphosphate), dCTP(2′-deoxycytidine-5′-triphosphate), dGTP(2′-deoxyguanosine-5′-triphosphate) and dTTP(2′-deoxythymidine-5′-triphosphate). The amount of the reaction solution was 20 μL.

With respect to each of six types of bacteria, forty-six types of reaction solutions for SSC-PCR containing primers of sequence Nos. 1 to 46, respectively were prepared simultaneously. Further, as a positive control, a reaction solution that contains the primer of sequence No. 14 and template DNA having a base sequence corresponding to that of the primer of sequence No. 14, and as a negative control, a reaction solution that contains the primer of sequence No. 14 but not DNA were prepared at the same time as the preparation of the forty-six types of reaction solutions for SSC-PCR.

Then, the forty-six types of SSC-PCR reaction solutions prepared as above were accommodated, respectively, in forty-six openings 51 of the DNA fragment amplifying apparatus shown in FIG. 4, while the negative control and the positive control were accommodated in the openings 51a and 51b, respectively. The SSC-PCR was carried out by employing this DNA fragment amplifying apparatus.

The cycles of SSC-PCR are shown in Table 11.

TABLE 11

| | |
|---|---|
| 94° C. for 1 min. | 1 cycle |
| 94° C. for 1 min. + 45° C. for 2 min. + 72° C. for 3 min. | 35 cycles |
| 72° C. for 7 min. | 1 cycle |
| 4° C. (end of reaction, preserved) | |

Then, 5 μL of each of the reaction solutions, the positive control and the negative control was analyzed by 1.5% agarose gel electrophoresis after SSC-PCR. The electrophoresis was made under the condition of a 3.6 V/cm constant voltage. In this case, a quantitatively analyzable DNA size marker (Smart Ladder manufactured by Nippon Gene Co., Ltd.) that has a known concentration was electrophoresed simultaneously with the reaction solutions in order to measure the position of bands of the amplified DNA fragments.

After the electrophoresis, gels were stained with ethidium bromide, and ethidium bromide fluorescent images obtained when irradiated with ultraviolet rays of 254 nm in wavelength were photographed by a Polaroid camera or a CCD camera.

In the electrophoresis of the positive control, the negative control and the 46 types of SSC-PCR reaction solutions, eight types of samples were simultaneously electrophoresed in a single sheet of agarose gel and were photographed for each agarose gel. Thus, a total of six electrophoretic photographs (Nos. 1 to 6) were obtained. In this case, the DNA size marker was electrophoresed on the opposite ends of each agarose gel.

After those six electrophoretic photographs (Nos. 1 to 6) were captured in the computer by the scanner, the band of the DNA size marker, the band in each primer and those in the positive control and the negative control were detected from the image data of the electrophoretic images with employment of software (Genomic Solutions Advanced Quantifier 1-D Match).

At that time, no band was detected in the electrophoretic image of the negative control. This made it possible to confirm that appearing bands were derived from the bacteria employed as samples.

The following step of image correction may further be carried out although such a step was not carried out in the above case. As image correction, it is considered that the luminous intensity of another band is corrected on the basis of the luminous intensity of the band in the positive control. If the measured luminous intensity of the band in the positive control is 70%, for example, this luminous intensity is corrected to 100% while the intensity of another band is also corrected in the same proportion as that of the positive control band. This makes it possible to eliminate any influences exerted to the amplification efficiency of DNA fragments by errors in reaction conditions and the like in the amplification and reaction of DNA fragments.

Alternatively, it is considered that in the image data of each electrophoretic image, the measured luminous intensity of a 1000 bp band in the DNA size marker is corrected in such a proportion as to be 100%, while the luminous intensity of another band is also corrected in the same proportion as that of the 1000 bp band in the DNA size marker. Thus, it is made possible to eliminate errors in gradation in each image data by correcting the gradation of the image data.

It is also considered that a half of the measured luminous intensity of a 200 bp band of the DNA size marker is determined as a threshold to eliminate those of bands in each primer whose luminous intensities are smaller than the threshold. It is thus made possible to eliminate the bands with lower reproducibility in SSC-PCR by eliminating the bands with smaller luminous intensities. This enables enhanced reproducibility in SSC-PCR and increased reliability of the obtained data.

\mathring{4} Measurement of Band Intensity and Band Position

The DNA size marker employed as above is separated into fourteen bands of different band sizes (the number of base pairs) by electrophoresis. The sizes of those fourteen bands are as follows in descending order: 10000, 8000, 6000, 5000, 4000, 3000, 2500, 2000, 1500, 1000, 800, 600, 400 and 200 bp. In this case, however, the 10000 bp band and the 8000 bp band are not separated since electrophoresis is made by employing a 1.5% agarose gel. Thus, thirteen bands appear. Spacings between thirteen bands are not uniform, but as the sizes of the bands increase, the spacings between the bands decrease.

As a method of displaying the positions of bands in each primer, there is a method of comparing the positions of bands in the DNA size marker with those in each primer, so as to convert the band positions in each primer into the band sizes (the number of base pairs) for displaying. This method, however, causes the following problems.

With respect to the band around 200 bp, for example, if the position of the band on the electrophoretic image is different by 1 mm, then the band size is different by 40 bp. In contrast, with respect to the band around 5000 bp, if the position of the band on the electrophoretic image is different by 1 mm, then the band size is different by 1000 bp. Thus, while 1 mm corresponds to 1000 bp as for the band around 5000 bp, 1 mm corresponds to 40 bp as for the band around 200 bp. The resolution of the image data of the electrophoretic image captured by the scanner is at most sub mm.

As has been described in the foregoing, the bands of larger sizes provide larger errors in the case where the band position in each primer is displayed by the band size (the number of base pairs). Therefore, in this case, the size of errors is required to be changed depending on the band size.

From the foregoing, it is more preferable to display the band position obtained in each primer on the basis of a measured value in the direction of an electrophoretic distance rather than displaying the band position by the band size since the sizes of errors in band positions become constant.

In electrophoresis, the electrophoretic distance varies depending on the time and the temperature in electrophoresis. Thus, standardization is required in order to display the band position on the basis of the measured value in the direction of electrophoretic distance in electrophoresis. A method of standardization will be described below.

For standardization, the DNA size marker is newly subjected to electrophoresis, and twelve electrophoretic photographs made by photographing the electrophoretic image of the DNA size marker were prepared.

First, with respect to each of the image data of those twelve electrophoretic photographs (Nos. 1 to 12), in reference to a 1000 bp band of the DNA size marker, the distances between the reference 1000 bp band and other twelve bands were measured. The results of measurement are shown in Table 12.

TABLE 12

| | Measured Distance from 1000 bp (unit: cm) | | | | | | | | | | | |
|---|---|---|---|---|---|---|---|---|---|---|---|---|
| | Photo- | Photo- | Photo- | Photo- | Photo- | Photo- | Photo- | Photo- | Photo- | Photo- | Photo- | Photo- |
| Band/bp | graph1 | graph2 | graph3 | graph4 | graph5 | graph6 | graph7 | graph8 | graph9 | graph10 | graph11 | graph12 |
| 10000-8000 | 1.66 | 1.61 | 1.64 | 1.65 | 1.68 | 1.57 | 1.66 | 1.66 | 1.65 | 1.68 | 1.69 | 1.63 |
| 6000 | 1.57 | 1.52 | 1.55 | 1.57 | 1.60 | 1.49 | 1.59 | 1.59 | 1.57 | 1.60 | 1.59 | 1.52 |
| 5000 | 1.50 | 1.47 | 1.49 | 1.50 | 1.54 | 1.42 | 1.50 | 1.50 | 1.49 | 1.51 | 1.51 | 1.45 |
| 4000 | 1.38 | 1.38 | 1.38 | 1.41 | 1.42 | 1.33 | 1.40 | 1.40 | 1.38 | 1.41 | 1.41 | 1.36 |
| 3000 | 1.23 | 1.21 | 1.23 | 1.23 | 1.27 | 1.17 | 1.23 | 1.22 | 1.21 | 1.22 | 1.23 | 1.18 |
| 2500 | 1.09 | 1.07 | 1.05 | 1.08 | 1.10 | 1.02 | 1.07 | 1.07 | 1.04 | 1.05 | 1.07 | 1.03 |
| 2000 | 0.88 | 0.84 | 0.85 | 0.86 | 0.89 | 0.81 | 0.85 | 0.84 | 0.84 | 0.84 | 0.84 | 0.83 |
| 1500 | 0.51 | 0.49 | 0.51 | 0.51 | 0.53 | 0.48 | 0.51 | 0.49 | 0.49 | 0.49 | 0.51 | 0.49 |
| 1000 | 0.00 | 0.00 | 0.00 | 0.00 | 0.00 | 0.00 | 0.00 | 0.00 | 0.00 | 0.00 | 0.00 | 0.00 |
| 800 | −0.29 | −0.27 | −0.28 | −0.28 | −0.28 | −0.27 | −0.27 | −0.28 | −0.25 | −0.28 | −0.27 | −0.25 |
| 600 | −0.66 | −0.65 | −0.64 | −0.64 | −0.65 | −0.60 | −0.61 | −0.62 | −0.62 | −0.64 | −0.60 | −0.60 |
| 400 | −1.05 | −1.07 | −1.05 | −1.09 | −1.05 | −1.03 | −1.03 | −1.07 | −1.02 | −1.07 | −1.02 | −1.02 |
| 200 | −1.61 | −1.63 | −1.59 | −1.66 | −1.60 | −1.59 | −1.57 | −1.61 | −1.55 | −1.65 | −1.56 | −1.56 |

In this case, since a 8000 bp band and a 10000 bp band are not separated, those bands are treated as one band of 8000 to 10000 bp as described above. Further, the measured distance of a 1000 bp band is set to 0.00, and the measured distance of each band of a smaller size than the 1000 bp band is represented by a negative value.

Then, with respect to each of the image data of the twelve photographs (Nos. 1 to 12), in reference to the measured distance between the 1000 bp band and the 10000 bp band being set to 1, the positions of other bands were standardized.

In the data of the photograph No. 1, for example, the measured distance between the 1000 bp band and the 10000 bp band is 1.66 cm. Accordingly, the measured distance values of other bands were divided by the value 1.66 for standardization of the respective positions of the other bands. The same operation was also carried out for the data of the remaining photographs Nos. 2 to 12. The results are shown in Table 13.

TABLE 13

| | Relative Position(Ratio) from 1000 bp | | | | | | | | | | | | Mean of |
|---|---|---|---|---|---|---|---|---|---|---|---|---|---|
| | Photo- | Photo- | Photo- | Photo- | Photo- | Photo- | Photo- | Photo- | Photo- | Photo- | Photo- | Photo- | Relative |
| Band/bp | graph1 | gaph2 | graph3 | graph4 | graph5 | graph6 | graph7 | graph8 | graph9 | graph10 | graph11 | graph12 | Position |
| 10000-8000 | 1.00 | 1.00 | 1.00 | 1.00 | 1.00 | 1.00 | 1.00 | 1.00 | 1.00 | 1.00 | 1.00 | 1.00 | 1.00 |
| 6000 | 0.95 | 0.94 | 0.95 | 0.95 | 0.95 | 0.95 | 0.96 | 0.96 | 0.95 | 0.95 | 0.94 | 0.93 | 0.95 |
| 5000 | 0.90 | 0.91 | 0.91 | 0.91 | 0.92 | 0.90 | 0.90 | 0.90 | 0.90 | 0.90 | 0.89 | 0.89 | 0.90 |
| 4000 | 0.83 | 0.86 | 0.84 | 0.85 | 0.85 | 0.85 | 0.84 | 0.84 | 0.84 | 0.84 | 0.83 | 0.83 | 0.84 |
| 3000 | 0.74 | 0.75 | 0.75 | 0.75 | 0.76 | 0.75 | 0.74 | 0.73 | 0.73 | 0.73 | 0.73 | 0.72 | 0.74 |
| 2500 | 0.66 | 0.66 | 0.64 | 0.65 | 0.65 | 0.65 | 0.64 | 0.64 | 0.63 | 0.63 | 0.63 | 0.63 | 0.64 |
| 2000 | 0.53 | 0.52 | 0.52 | 0.52 | 0.53 | 0.52 | 0.51 | 0.51 | 0.51 | 0.50 | 0.50 | 0.51 | 0.51 |
| 1500 | 0.31 | 0.30 | 0.31 | 0.31 | 0.32 | 0.31 | 0.31 | 0.30 | 0.30 | 0.29 | 0.30 | 0.30 | 0.30 |
| 1000 | 0.00 | 0.00 | 0.00 | 0.00 | 0.00 | 0.00 | 0.00 | 0.00 | 0.00 | 0.00 | 0.00 | 0.00 | 0.00 |
| 800 | −0.18 | −0.17 | −0.18 | −0.17 | −0.18 | −0.17 | −0.17 | −0.17 | −0.16 | −0.17 | −0.17 | −0.16 | −0.17 |
| 600 | −0.41 | −0.40 | −0.40 | −0.39 | −0.41 | −0.38 | −0.39 | −0.39 | −0.40 | −0.39 | −0.38 | −0.38 | −0.39 |
| 400 | −0.65 | −0.66 | −0.66 | −0.66 | −0.66 | −0.65 | −0.66 | −0.66 | −0.66 | −0.65 | −0.65 | −0.65 | −0.66 |
| 200 | −1.00 | −1.00 | −1.00 | −1.00 | −1.00 | −1.00 | −1.00 | −1.00 | −1.00 | −1.00 | −1.00 | −1.00 | −1.00 |

Further, as shown in Table 13, a mean in the data of twelve electrophoretic photographs (Nos. 1 to 12) was evaluated as for the bands of each size. As described above, in reference to the position of the 1000 bp band in the DNA size marker, the relative positions of the other bands (ratios) were evaluated.

In addition, the position of the reference 1000 bp band was set to 1000, and the position of the 8000 to 10000 bp band whose relative distance from 1000 bp is 1.00 was set to 2000 in order to facilitate viewing of the above obtained values. On the basis of these values for band position, the band positions of the remaining bands were derived. Thus, the band positions (reference values) as to the thirteen bands of the DNA size marker were evaluated. The results were shown in Table 14.

TABLE 14

| | 10000- | | | | | | | | | | | | |
|---|---|---|---|---|---|---|---|---|---|---|---|---|---|
| Band/bp | 8000 | 6000 | 5000 | 4000 | 3000 | 2500 | 2000 | 1500 | 1000 | 800 | 600 | 400 | 200 |
| Means of Relative | 1.00 | 0.95 | 0.90 | 0.84 | 0.74 | 0.64 | 0.51 | 0.30 | 0.00 | −0.17 | −0.38 | −0.64 | −0.97 |
| Position | | | | | | | | | | | | | |
| Reference | 2000 | 1948 | 1904 | 1842 | 1740 | 1644 | 1514 | 1304 | 1000 | 830 | 620 | 360 | 30 |
| Value | | | | | | | | | | | | | |

While the above description has been made on the standardization method in which the position of the 1000 bp band of the DNA size marker is set as a reference, while the position of the 8000 to 10000 bp band is set to 1, another standardization method may be applicable.

Then, the band position and the band intensity in each of forty-six types of primers were measured with respect to each of six types of bacteria on the basis of the band positions (reference values) of the DNA size marker as obtained above. Further, a list of band data was created for each bacterium by collecting and summing up the data obtained by measurement. In this case, the height of peaks in luminous distributions of bands was set as the band intensity.

In this case as described above, with respect to each band of the image data of the electrophoretic image, no correction was made on the luminous intensities of other bands on the basis of the luminous intensity of the band in the positive control; no gradient correction was made based on the luminous intensity of the DNA size marker; or no elimination was made for bands whose luminous intensities were less than the threshold.

The foregoing software (Genomic Solutions Advanced Quantifier 1-D Match) was employed for the measurement of such band positions and intensities. A software created in Microsoft Visual Basic by the inventor of the present invention was employed for the step of making a list by totaling the data of band positions and band intensities of image data of six electrophoretic photographs.

Tables 15 to 17, for example, show a list of band data with respect to the bacterium No. 2001.

TABLE 15

| Primer No. | Band Position | Band Intensity |
|---|---|---|
| 1 | 1903.8 | 0.13 |
| 1 | 1539.7 | 0.98 |
| 1 | 1003.4 | 0.10 |
| 2 | 1652.2 | 0.24 |
| 3 | 1677.7 | 0.93 |
| 3 | 1493.2 | 0.19 |
| 3 | 1058.8 | 0.82 |
| 4 | 1367.2 | 0.13 |
| 4 | 965.8 | 0.31 |
| 4 | 198.8 | 0.09 |
| 6 | 567.9 | 0.23 |
| 6 | 250.9 | 0.87 |
| 8 | 1815.4 | 0.19 |
| 8 | 1211.8 | 0.59 |
| 8 | 809.6 | 0.46 |
| 8 | 701.7 | 0.60 |
| 9 | 1832.2 | 0.10 |
| 9 | 1459.8 | 0.69 |
| 9 | 1288.2 | 0.15 |
| 9 | 1191.8 | 0.71 |
| 9 | 984.4 | 0.19 |
| 9 | 799.7 | 0.13 |
| 10 | 1847.8 | 0.09 |
| 10 | 1265.2 | 0.22 |
| 10 | 1201.1 | 0.34 |
| 10 | 385.2 | 0.07 |
| 11 | 1606.5 | 0.21 |
| 11 | 1363.6 | 0.07 |
| 12 | 1677.1 | 0.13 |
| 12 | 1447.5 | 0.51 |
| 12 | 1036.2 | 1.01 |
| 12 | 932.8 | 0.12 |
| 12 | 88 | 0.23 |
| 13 | 1471.7 | 0.11 |
| 13 | 743.8 | 0.09 |
| 14 | 1931 | 0.09 |
| 14 | 1587.9 | 0.40 |
| 14 | 1442 | 0.76 |
| 14 | 1325 | 0.78 |
| 14 | 1145.5 | 0.53 |
| 14 | 1037.9 | 0.78 |
| 14 | 694.1 | 0.29 |
| 14 | 564.8 | 0.45 |
| 14 | 416.8 | 0.11 |
| 15 | 1629.3 | 0.10 |
| 15 | 1473.8 | 0.12 |
| 16 | 1251.3 | 0.13 |
| 16 | 960.3 | 0.09 |
| 16 | 865.9 | 0.58 |
| 16 | 629.2 | 0.07 |
| 16 | 543.9 | 0.18 |
| 17 | 1286.5 | 0.82 |
| 18 | 1970 | 0.18 |
| 18 | 1706.9 | 0.38 |
| 18 | 1579.4 | 1.14 |
| 18 | 1127.5 | 0.20 |
| 18 | 1021.3 | 0.17 |
| 20 | 1737 | 0.51 |
| 20 | 1435.4 | 0.19 |
| 20 | 910.2 | 0.15 |

TABLE 16

| Primer No. | Band Position | Band Intesity |
|---|---|---|
| 21 | 1768.1 | 0.53 |
| 21 | 1490.9 | 0.35 |
| 21 | 1003.6 | 0.61 |
| 21 | 563.2 | 0.15 |
| 22 | 1602 | 0.37 |
| 22 | 1513.3 | 0.83 |
| 22 | 1389.5 | 0.21 |
| 22 | 1345.1 | 0.14 |
| 22 | 1213.3 | 0.13 |
| 22 | 1114.7 | 0.76 |
| 22 | 905.5 | 0.12 |
| 22 | 775.3 | 0.19 |
| 22 | 648.9 | 0.22 |
| 22 | 573.1 | 0.12 |
| 23 | 1137.2 | 0.11 |
| 24 | 1490.9 | 0.29 |
| 24 | 1123 | 0.24 |
| 24 | 1032.3 | 0.13 |
| 24 | 908.2 | 0.19 |
| 24 | 803.6 | 0.49 |
| 24 | 157.3 | 0.28 |
| 25 | 1482.1 | 0.79 |
| 26 | 1481.3 | 0.80 |
| 26 | 1316.1 | 1.18 |
| 26 | 879.4 | 0.13 |
| 27 | 1418.4 | 0.20 |
| 27 | 1246.5 | 1.16 |
| 27 | 1192.3 | 0.88 |
| 27 | 1023.6 | 1.07 |
| 27 | 692.6 | 0.19 |
| 27 | 608 | 0.39 |
| 27 | 435.3 | 0.60 |
| 29 | 1161.7 | 0.14 |
| 29 | 120.8 | 0.39 |
| 30 | 1734.9 | 0.43 |
| 30 | 1581.8 | 0.41 |
| 30 | 1462.4 | 0.19 |
| 30 | 1254.1 | 0.29 |
| 30 | 1191.5 | 0.20 |
| 30 | 834.3 | 0.19 |
| 30 | 702.7 | 1.20 |
| 30 | 105.5 | 0.12 |
| 31 | 1262.1 | 0.17 |
| 31 | 993.8 | 0.90 |
| 31 | 536 | 1.09 |
| 31 | 427.7 | 0.98 |
| 32 | 1315.5 | 0.30 |
| 32 | 791 | 0.36 |
| 33 | 1872.8 | 0.09 |
| 33 | 1615.5 | 0.21 |
| 33 | 1482.2 | 0.39 |
| 33 | 1411.9 | 0.38 |
| 33 | 1160.8 | 0.80 |
| 33 | 1027.3 | 0.43 |
| 33 | 945.4 | 0.25 |
| 33 | 885.2 | 0.30 |
| 34 | 1785.3 | 0.39 |
| 34 | 1512.5 | 0.89 |
| 34 | 1359.2 | 0.21 |
| 34 | 1238.3 | 0.17 |

TABLE 17

| Primer No. | Band Position | Band Intensity |
|---|---|---|
| 34 | 1139.5 | 0.26 |
| 34 | 1089.6 | 0.42 |
| 34 | 1022 | 0.16 |
| 34 | 873.6 | 0.21 |
| 34 | 778.8 | 0.22 |
| 34 | 716 | 0.29 |
| 35 | 1330.1 | 0.56 |
| 35 | 1133.1 | 0.64 |
| 36 | 1270.7 | 0.39 |
| 36 | 1005.5 | 0.28 |
| 36 | 907.8 | 1.42 |
| 36 | 390.8 | 0.91 |
| 37 | 1365.6 | 0.37 |
| 38 | 1826 | 0.26 |
| 38 | 1687.4 | 0.31 |
| 38 | 1568.3 | 1.14 |
| 38 | 1424.3 | 1.15 |
| 38 | 1247.3 | 0.80 |
| 38 | 971.8 | 0.31 |
| 38 | 724.4 | 0.65 |
| 38 | 639.8 | 0.43 |
| 38 | 416.2 | 1.13 |
| 39 | 1800 | 0.96 |
| 40 | 1588.3 | 0.66 |
| 40 | 1430.7 | 0.16 |
| 40 | 1095.8 | 0.90 |
| 40 | 786.3 | 0.20 |
| 40 | 477.3 | 0.19 |
| 40 | 214 | 0.89 |
| 41 | 1193.5 | 0.10 |
| 41 | 131.1 | 0.21 |
| 42 | 1092.5 | 0.59 |
| 42 | 896.7 | 0.43 |
| 42 | 148 | 0.11 |
| 43 | 1595.2 | 0.34 |
| 44 | 1548.5 | 0.20 |
| 44 | 1422.1 | 0.32 |
| 44 | 1145.7 | 0.59 |
| 44 | 888 | 0.28 |
| 44 | 818.5 | 0.31 |
| 45 | 1644.1 | 0.37 |
| 45 | 1533.8 | 0.38 |
| 45 | 1302.2 | 0.58 |
| 45 | 1153.6 | 0.18 |
| 45 | 835. 1 | 0.23 |
| 45 | 675.6 | 0.61 |
| 46 | 1722.4 | 0.37 |
| 46 | 1645 | 0.60 |
| 46 | 1511.7 | 0.57 |
| 46 | 1161.4 | 0.40 |
| 46 | 808 | 0.36 |
| 46 | 573.5 | 0.48 |

such a list of band data was made with respect also to each of the other five bacteria Nos. 1028, 1030, 4004, 5063 and 7004.

\mathring{5} Registration of Band Data List of Bacteria into Database

The band data list of the bacterium No. 2001 shown in Tables 15 to 17 above was registered in the database as data for searching. Similarly, the band data lists of the other five types of bacteria Nos. 1028, 1030, 4004, 5063 and 7004 were registered in the database as data for searching.

2. Data Searching when Each of Six Types of Single Isolated Bacteria are Employed as Samples

In order to test searching when a single bacterium was used as a sample, the DNA analysis was again made on each of those isolated bacteria Nos. 1028, 1030, 4000, 5063, 7004 and 2001 by the foregoing SSC-PCR, and searching was then carried out from the above created database on the basis of the resultant sample data.

As for the method of DNA analysis and the method of measuring band intensities and band positions in that case, these methods are the same as those described in the steps \mathring{3} and \mathring{4}× in the creation of database. In this case also, the above-described software (Genomic Solutions Advanced Quantifier 1-D Match) and the software created by the inventor of the present invention were employed.

In this case, after the band data list was made for each of the six types of bacteria as samples, the created band data of each sample bacterium was compared with the band data of the six types of bacteria that was previously registered in the database, so that correlation coefficients were evaluated.

The calculation of correlation coefficients was made by employing the software created in Microsoft Visual Basic by the inventor of the present invention. As to parameters in this case, an error in band positions (the range of band position in which bands are considered to be on the same position) was set to ±43, and a threshold of correlation coefficients was set to 0.30. As a method of calculating correlation coefficients, the method shown in FIG. 12(c) was employed. As the result of calculation of correlation coefficients as described above, such a result as shown in Table 18 was obtained.

TABLE 18

| | Database | | | | | |
|---|---|---|---|---|---|---|
| Sample | 1028 | 1030 | 4004 | 5063 | 7004 | 2001 |
| 1028 | 0.692 | −0.104 | 0.113 | 0.204 | −0.165 | −0.020 |
| 1030 | 0.142 | 0.667 | 0.167 | 0.037 | −0.143 | 0.040 |
| 4004 | 0.082 | 0.006 | 0.675 | 0.147 | −0.047 | 0.044 |
| 5063 | 0.101 | 0.097 | 0.262 | 0.699 | −0.225 | −0.008 |
| 7004 | 0.036 | −0.049 | 0.074 | −0.112 | 0.631 | 0.026 |
| 2001 | 0.087 | 0.086 | 0.11 | 0.002 | −0.175 | 0.906 |

When six types of single bacteria were employed as samples, those sample bacteria were able to be searched from the database, as shown in Table 18.

The following was made apparent from the result of analysis shown in Table 18. That is, if it is already known that the sample for use is a single bacterium, then it is made possible to securely search for the sample bacterium from the database by setting the threshold of correlation coefficients to about 0.6. On the other hand, as to the correlation coefficients among different types of bacteria, the correlation coefficient between the bacterium No. 5063 as the sample and the bacterium No. 4004 is 0.252, which is the greatest value, and the other coefficients are small. From this relationship, it is found that erroneous ones are not searched if the threshold is set to not less than 0.3.

In addition, a correlation state between band data of the bacterium No. 2001 used as the sample and that of the bacterium No. 2001 registered in the database was confirmed on the basis of a correlation diagram. The result is shown in FIG. 14. The straight line in FIG. 14 indicates the case where the correlation coefficient is 1, i. e., both data match each other.

With reference to FIG. 14, it is found that there is a highly excellent correlation between the band data of the sample bacterium No. 2001 and that of the database bacterium No. 2001. Thus, it is made possible to visually confirm the excellent correlation of both data by exhibiting such a correlation diagram as shown in FIG. 14.

3. Data Searching when Mixture of Plurality of Bacteria are Employed as Sample

In order to test searching in the case where a mixture of a plurality of bacteria was used as a sample, a sample 1 including five types of bacteria (Nos. 1028, 1030, 4004, 5063, and 7004) and a sample 2 including two types of bacteria (Nos. 1028 and 1030) were prepared. A DNA analysis was made on each of the samples 1 and 2 by the foregoing SSC-PCR, and searching was then carried out from the above created database on the basis of data of the samples 1 and 2 obtained by the DNA analysis.

When the sample 1 was employed for SSC-PCR, a reaction solution for SSC-PCR containing chromosome DNA of the five types of bacteria each at a concentration of 1 μg/L was prepared. When the sample 2 was employed for SSC-PCR, a reaction solution for SSC-PCR containing chromosome DNA of the two types of bacteria each at a concentration of 1 μg/L was prepared.

The same method as the one described above in the steps \mathring{3} and \mathring{4} in the creation of database was applied except the method of preparation of the above reaction solutions for SSC-PCR. In this case also, the foregoing software (Genomic Solutions Advanced Quantifier 1-D Match) and the software created by the inventor of the present invention were employed.

In this case, a list of band data was created with respect to each of the samples 1 and 2. After that, the created band data of the samples 1 and 2 were compared with the band data of six types of bacteria that were previously registered in the database, so as to evaluate correlation coefficients.

Calculation of the correlation coefficients was carried out by employing the above-described software created by the inventor of the present invention. As to parameters in this case, an error in band position (the range of band position in which bands are considered to be on the same position) was set to ±43, and a threshold of correlation coefficients was set to 0.30. As a method of calculating correlation coefficients, the method shown in FIG. 12 (c) was employed. As the result of calculation of correlation coefficients as described above, such a result as shown in Table 19 was obtained.

TABLE 19

| | Database | | | | | |
|---|---|---|---|---|---|---|
| Sample | 1028 | 1030 | 4004 | 5063 | 7004 | 2001 |
| Sample1 | 0.515 | 0.426 | 0.613 | 0.290 | 0.585 | 0.073 |
| Sample2 | 0.552 | 0.454 | 0.104 | 0.314 | −0.077 | 0.010 |

As shown in Table 19, with respect to the sample 1 including the five types of bacteria, four types of bacteria Nos. 1028, 1030, 4004 and 7004 of those five mixed types were searched from the database. Since the threshold was set to 0.30, the bacterium No. 5063 included in the sample 1 was not searched in this case. This result made it clear that the threshold 0.3 was high for the bacterium No. 5063 with respect to the sample 1.

On the other hand, with respect to the sample 2 including the two mixed types of bacteria, those bacteria Nos. 1028 and 1030 were searched from the database. In this case, the bacterium No. 5063 not included in the sample 2 was searched. This result made it clear that the threshold 0.3 was low for the bacterium No. 5063 with respect to the sample 2.

The above results of analysis indicate that it is possible to securely search for the bacteria included in the samples if the threshold for searching is set to a higher value, e.g. 0.35, while it is possible to widely search for the bacteria that can be included in the samples if the threshold for searching is set to a lower value, e.g., 0.25.

1. A microorganism discriminating method that discriminates a target microorganism to be discriminated, comprising the steps of:
preparing a plurality of primers having different amplification probabilities, and with employment of each of said plurality of primers, applying at a time a polymerase chain reaction method in which a thermal denaturation step, a primer annealing step and an extension reaction step with polymerase are repeated in this order, to DNA of said target microorganism to be discriminated, thereby amplifying DNA fragments of the DNA of said target microorganism to be discriminated;
applying electrophoresis to the DNA fragments amplified by employing each of said plurality of primers, to obtain an electrophoretic image corresponding to each primer;
converting said electrophoretic image corresponding to each primer into image data;
detecting a band of said electrophoretic image corresponding to each primer on the basis of said image data;
finding information as to a position and an intensity of said detected band in said electrophoretic image corresponding to each primer on the basis of said image data;
finding a correspondence between said plurality of primers and the information as to the position and the intensity of said band;
searching database in which said correspondence with respect to a microorganism to be collated with is stored previously, to find a correlation between said correspondence found with respect to said target microorganism to be discriminated and said correspondence with respect to said microorganism to be collated with; and
discriminating said target microorganism to be discriminated on the basis of said correlation.

2. The microorganism discriminating method as recited in claim 1, wherein the information as to the position of said band is expressed by a distance between a predetermined reference position on said electrophoretic image and said band.

3. The microorganism discriminating method as recited in claim 1, wherein the information as to the position of said band is expressed by the size of DNA fragments included in said band.

4. The microorganism discriminating method as recited in claim 1, wherein the information as to the intensity of said band expresses the height or area of a peak in luminous intensity distribution of the band in said electrophoretic image and is calculated on the basis of gradient distribution of said image data.

5. The microorganism discriminating method as recited in claim 1, wherein the correspondence between said plurality of primers and the information as to the position and the intensity of said band indicates for each primer a correspondence between information as to the position of each band and information as to the intensity of each band.

6. The microorganism discriminating method as recited in claim 1, wherein said correlation represents a correlation between the intensities of bands on an approximately identical position with respect to said target microorganism to be discriminated and said microorganism to be collated with.

7. The microorganism discriminating method as recited in claim 1, wherein said step of discriminating said target microorganism to be discriminated on the basis of said correlation includes the step of calculating a correlation coefficient in said correlation to determine whether said target microorganism is said microorganism to be collated with on the basis of whether or not said calculated correlation coefficient is larger than a predetermined threshold.

8. The microorganism discriminating method as recited in claim 1, further comprising the steps of:
with employment of a reference primer having a known base sequence, applying said polymerase chain reaction method to reference DNA having a base sequence complementary with the base sequence of said reference primer, thereby amplifying DNA fragments of said reference DNA;
applying electrophoresis to the DNA fragments of said reference DNA amplified by employing said reference primer, to obtain an electrophoretic image corresponding to said reference DNA;
converting said electrophoretic image corresponding to said reference DNA into image data; and
correcting image data corresponding to said target microorganism to be discriminated on the basis of the image data corresponding to said reference DNA.

9. The microorganism discriminating method as recited in claim 1, further comprising the steps of:
obtaining an electrophoretic image of a DNA size marker simultaneously with the step of obtaining the electrophoretic image corresponding to each primer;
converting the electrophoretic image of said DNA size marker into image data; and
correcting the image data corresponding to said target microorganism to be discriminated on the basis of the image data corresponding to said DNA size marker.

10. The microorganism discriminating method as recited in claim 9, wherein said step of detecting the band of said electrophoretic image includes the step of setting a threshold based on a luminous intensity of the band of the DNA fragments amplified from said reference DNA or a luminous intensity of a band of said DNA size marker in said electrophoretic image, to select a band having a luminous intensity not less than said threshold in said electrophoretic image.

11. The microorganism discriminating method as recited in claim 1, wherein said correspondence with respect to plural types of microorganisms is stored in said database.

12. A microorganism discriminating apparatus that discriminates a target microorganism to be discriminated, comprising:
first amplifying means for applying at a time a polymerase chain reaction method in which a thermal denaturation step, a primer annealing step and an extension reaction step with polymerase are repeated in this order, to DNA of said target microorganism to be discriminated, with employment of each of a plurality of primers having different amplification probabilities, thereby amplifying DNA fragments of the DNA of said target microorganism;
first electrophoresis means for applying electrophoresis to the DNA fragments amplified by said first amplifying means employing each of said plurality of primers, to obtain an electrophoretic image corresponding to each primer;
band detecting means for detecting a band of said electrophoretic image corresponding to each primer on the basis of said image data obtained by said first image data converting means;
band information detecting means for finding information as to a position and an intensity of said detected band in said electrophoretic image corresponding to each primer on the basis of said image data;
correspondence creating means for creating a correspondence between said plurality of primers and the information as to the position and the intensity of said band detected by said band information detecting means;
correlation creating means for searching database in which said correspondence with respect to a microorganism to be collated with is stored previously, to create a correlation between said correspondence created with respect to said target microorganism to be discriminated and said correspondence with respect to said microorganism to be collated with; and
discriminating means for discriminating said target microorganism on the basis of the correlation created by said correlation creating means.

13. The microorganism discriminating apparatus as recited in claim 12, further comprising:
second amplifying means for applying said polymerase chain reaction method, with employment of a reference primer having a known base sequence, to reference DNA having a base sequence complementary with the base sequence of said reference primer, thereby amplifying DNA fragments of said reference DNA;
second electrophoresis means for applying electrophoresis to the DNA fragments of said reference DNA amplified by employing said reference primer, to obtain an electrophoretic image corresponding to said reference DNA;
second image data converting means for converting said electrophoretic image corresponding to said reference DNA into image data; and
first correcting means for correcting image data corresponding to said target microorganism to be discriminated on the basis of the image data corresponding to said reference DNA.

14. The microorganism discriminating apparatus as recited in claim 12, further comprising:
third electrophoresis means for obtaining an electrophoretic image of a DNA size marker simultaneously with obtaining the electrophoretic image corresponding to each primer;
third image data converting means for converting the electrophoretic image of said DNA size marker into image data; and
second correcting means for correcting the image data corresponding to said target microorganism to be discriminated on the basis of the image data corresponding to said DNA size marker.

15. The microorganism discriminating apparatus as recited in claim 14, wherein said band detecting means includes band selecting means for setting a threshold based on a luminous intensity of the band of the DNA fragments amplified from said reference DNA or a luminous intensity of a band of said DNA size marker in said electrophoretic image, to select a band having a luminous intensity not less than said threshold in said electrophoretic image.

16. The microorganism discriminating apparatus as recited in claim 12, wherein said correspondence with respect to plural types of microorganisms is stored in said database.

17. A recording medium readable by a computer, on which a microorganism discriminating program for discriminating a target microorganism to be discriminated is stored, wherein
said microorganism discriminating program makes said computer execute the processings of:
applying at a time a polymerase chain reaction method in which a thermal denaturation step, a primer annealing step and an extension reaction step with polymerase are repeated in this order, to DNA of said target microorganism to be discriminated, with employment of each of a plurality of primers with different amplification probabilities, thereby amplifying DNA fragments of the DNA of said target microorganism to be discriminated, and applying electrophoresis to the DNA fragments amplified by employing each of said plurality of primers, so as to capture a resultant electrophoretic image as image data;
detecting a band of said electrophoretic image corresponding to each primer on the basis of said image data;
seeking information as to a position and an intensity of said detected band in said electrophoretic image corresponding to each primer on the basis of said image data;
finding a correspondence between said plurality of primers and the information as to the position and the intensity of said band;
searching database in which said correspondence with respect to a microorganism to be collated with is stored previously, to find a correlation between said correspondence found with respect to said target microorganism to be discriminated and said correspondence with respect to said microorganism to be collated with; and
discriminating said target microorganism on the basis of said correlation.

18. A method of creating database in which data of a microorganism to be collated with is stored in order to discriminate a microorganism, comprising the steps of:
preparing a plurality of primers having different amplification probabilities and, with employment of each of said plurality of primers, applying at a time a polymerase chain reaction method in which a thermal denaturation step, a primer annealing step and an extension reaction step with polymerase are repeated in this order, to DNA of a microorganism, thereby amplifying DNA fragments of the DNA of said microorganism;
applying electrophoresis to the DNA fragments amplified by employing each of said plurality of primers, to obtain an electrophoretic image corresponding to each primer;
converting said electrophoretic image corresponding to each primer into image data;
detecting a band of said electrophoretic image corresponding to each primer on the basis of said image data;
finding information as to a position and an intensity of said detected band in said electrophoretic image corresponding to each primer on the basis of said image data;
finding a correspondence between said plurality of primers and the information as to the position and the intensity of said band; and
storing in storing means said correspondence as data of said microorganism to be collated with.

19. A recording medium on which database storing therein data of a microorganism to be collated with in order to discriminate a microorganism is recorded, wherein
said database is created by a method of including the steps of:
preparing a plurality of primers having different amplification probabilities and, with employment of each of said plurality of primers, applying at a time a polymerase chain reaction method in which a thermal denaturation step, a primer annealing step and an extension reaction step with polymerase are repeated in this order, to DNA of a microorganism, thereby amplifying DNA fragments of the DNA of said microorganism;
applying electrophoresis to the DNA fragments amplified by employing each of said plurality of primers, to obtain an electrophoretic image corresponding to each primer;
converting said electrophoretic image corresponding to each primer into image data;
detecting a band of said electrophoretic image corresponding to each primer on the basis of said image data;
finding information as to a position and an intensity of said detected band in said electrophoretic image corresponding to each primer on the basis of said image data;
finding a correspondence between said plurality of primers and the information as to the position and the intensity of said band; and
storing in storing means said correspondence as data of said microorganism to be collated with.

20. A microorganism discriminating program readable by a computer, which discriminates a target microorganism to be discriminated, wherein
said program makes said computer execute the processings of:
applying at a time a polymerase chain reaction method in which a thermal denaturation step, a primer annealing step and an extension reaction step with polymerase are repeated in this order, to DNA of said target microorganism to be discriminated, with employment of each of a plurality of primers with different amplification probabilities, thereby amplifying DNA fragments of the DNA of said target microorganism to be discriminated, and applying electrophoresis to the DNA fragments amplified employing each of said plurality of primers, so as to capture a resultant electrophoretic image as image data;
detecting a band of said electrophoretic image corresponding to each primer on the basis of said image data;
finding information as to a position and an intensity of said detected band in said electrophoretic image corresponding to each primer on the basis of said image data;
finding a correspondence between said plurality of primers and the information as to the position and the intensity of said band;
searching database in which said correspondence with respect to a microorganism to be collated with is stored previously, to find a correlation between said correspondence found with respect to said target microorganism to be discriminated and said correspondence with respect to said microorganism to be collated with; and
discriminating said target microorganism on the basis of said correlation.
